(19) United States
(12) Patent Application Publication
(10) Pub. No.: US 2025/0345318 A1
(43) Pub. Date: Nov. 13, 2025

(54) COMPOUND AND USE THEREOF IN PREVENTING OR TREATING RADIATION DAMAGE

(71) Applicants: SIMCERE PHARMACEUTICAL CO., LTD., Nanjing, Jiangsu (CN); JIANGSU SIMCERE PHARMACEUTICAL CO., LTD., Nanjing, Jiangsu (CN)

(72) Inventors: Jianwei Zhou, Nanjing, Jiangsu (CN); Yan Zhou, Nanjing, Jiangsu (CN); Jingwen Liu, Nanjing, Jiangsu (CN); Xiong Li, Nanjing, Jiangsu (CN); Aiping Li, Nanjing, Jiangsu (CN); Feng Tang, Shanghai (CN); Lirong Hu, Shanghai (CN); Yuxi Yan, Shanghai (CN); Tengfei Xue, Nanjing, Jiangsu (CN); Yinqiang Zhuang, Nanjing, Jiangsu (CN); Yuanbin Hao, Shanghai (CN)

(73) Assignees: SIMCERE PHARMACEUTICAL CO., LTD., Nanjing, Jiangsu (CN); JIANGSU SIMCERE PHARMACEUTICAL CO., LTD., Nanjing, Jiangsu (CN)

(21) Appl. No.: 18/868,852

(22) PCT Filed: May 25, 2023
(86) PCT No.: PCT/CN2023/096248

§ 371 Date: Nov. 25, 2024

(30) Foreign Application Priority Data
May 26, 2022 (CN) 202210582602.7

(51) Int. Cl. A61K 31/4439; A61K 45/06; A61P 39/06; A61P 43/00

(52) U.S. Cl. CPC A61K 31/4439; A61K 45/06; A61P 39/06; A61P 43/00

(57) ABSTRACT

The present invention provides a compound and use thereof, particularly a JWA gene agonist and use thereof in preventing or treating radiation damage. The compound, by means of activating JWA gene expression, enhances the DNA repair capacity of cells, and has a good elimination capacity for highly active oxygen species generated by X-ray radiation. The compound can also effectively inhibit cell apoptosis caused by radiation to reduce the damage, and further reduce the generation of free radicals to reduce the probability of radiation damage.

The present disclosure claims priority to the Chinese Patent Application No. 202210582602.7 filed with China National Intellectual Property Administration on May 26, 2022, and entitled “COMPOUND AND USE THEREOF IN PREVENTING OR TREATING RADIATION DAMAGE”, which is incorporated herein by reference in its entirety.

### TECHNICAL FIELD

The present disclosure relates to the technical field of pharmaceuticals. In particular, the present disclosure relates to a compound and use thereof, and further, the present disclosure relates to a JWA gene agonist and use thereof in preventing or treating a radiation-induced injury.

### BACKGROUND

Ionizing radiation is a common physical environmental stimulus, including γ rays and X-rays, neutrons, α and β particles, high-velocity electrons, high-velocity protons, and other ions that can ionize upon collision with a given biological or non-biological material. Living cells, after absorbing the energy of ionizing radiation, in one aspect directly interact with DNA, disrupting the atomic structure of the cells and thereby causing chemical and biological changes. In another aspect, through the indirect action of irradiation on water and the bystander effect, a series of reactive chemical substances that may damage nucleic acids, proteins, and lipids are produced.

Currently, among the radioprotective medications approved by the FDA, only two medications, granulocyte colony-stimulating factor (G-CSF) and granulocyte-macrophage colony-stimulating factor (GM-CSF), are available for the treatment of hematopoietic acute radiation syndrome resulting from radiation exposure. However, there are no approved radioprotective medications for the treatment of radiation syndromes affecting other parts of the body at present. Small molecule compounds can be administered orally in the form of tablets or powders and typically do not cause immune responses, resulting in good patient compliance. Therefore, the development of a small molecule compound with the effect of preventing or treating radiation-induced injury has important clinical value.

JWA, also known as ARL6IP5 (GenBank AF070523, 1998), is a gene that was discovered and cloned from a retinoic acid-induced bronchial epithelial cell differentiation model.

### SUMMARY

The present disclosure relates to use of a JWA gene agonist or a pharmaceutical composition thereof in preventing or treating a radiation-induced injury.

In some embodiments, the JWA gene agonist is selected from a compound of formula (I) or a pharmaceutically acceptable salt thereof,

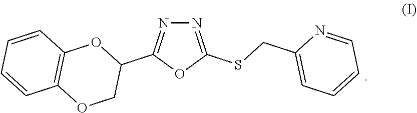 [SMILES: c1ccc(CSc2nnc(C3COc4ccccc4O3)o2)nc1]

In some embodiments, the compound of formula (I) is selected from one of R-JAC4 and S-JAC4, and a combination of the two, with the following structural formulas:

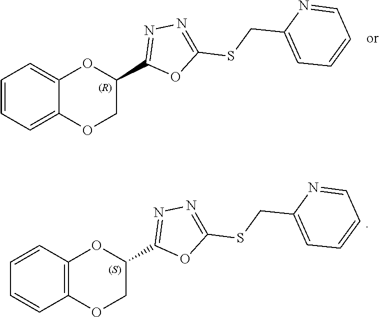

In some embodiments, the compound of formula (I) is selected from R-JAC4, with the following structural formula:

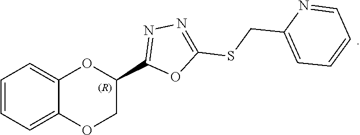

In some embodiments, the radiation-induced injury is selected from one or two of a radiation-induced intestinal injury and a radiation-induced lung injury.

In some embodiments, the radiation-induced injury is a radiation-induced intestinal injury.

In some embodiments, the radiation-induced intestinal injury is radiation enteritis.

In some embodiments, the radiation-induced injury is a radiation-induced lung injury.

In some embodiments, the radiation-induced lung injury is radiation pneumonitis.

In some embodiments, the radiation-induced lung injury is one or two of a radiation oxidative stress injury and a radiation DNA injury.

In some embodiments, the radiation-induced injury is selected from an X-ray radiation-induced injury.

In another aspect, the present disclosure provides a method for preventing or treating a radiation-induced injury, comprising: administering to a mammal, preferably a human, in need a therapeutically effective amount of a JWA gene agonist or a pharmaceutical composition thereof.

In another aspect, the present disclosure provides use of a JWA gene agonist or a pharmaceutical composition thereof in preparing a medicament for preventing or treating a radiation-induced injury.

In another aspect, the present disclosure provides a JWA gene agonist or a pharmaceutical composition thereof for preventing or treating a radiation-induced injury.

In some embodiments, the pharmaceutical composition comprises the JWA gene agonist and a pharmaceutically acceptable excipient.

In another aspect, the present disclosure provides use of a JWA gene agonist or a pharmaceutical composition thereof in preparing a medicament for preventing or treating a cancer patient, wherein the medicament is administered in combination with a radiation therapy. In another aspect, the present disclosure provides a method for preventing or treating a cancer in a mammal, comprising: administering to a mammal, preferably a human, in need a JWA gene agonist or a pharmaceutical composition thereof and a radiation therapy.

In another aspect, the present disclosure provides use of a JWA gene agonist or a pharmaceutical composition thereof in combination with a radiation therapy in preventing or treating a cancer.

In another aspect, the present disclosure provides a combination of a JWA gene agonist or a pharmaceutical composition thereof and a radiation therapy for preventing or treating a cancer.

In some embodiments, the cancer is selected from lung cancer.

In some embodiments, the cancer is selected from non-small cell lung cancer.

In some embodiments, the cancer is selected from lung adenocarcinoma.

The compound of the present disclosure can enhance the DNA repair capabilities in cells, effectively scavenge highly reactive oxygen species generated by X-ray irradiation by activating JWA gene expression, effectively inhibit radiation-induced cell apoptosis and thus mitigate injury, and reduce the production of free radicals and the occurrence of radiation-induced injury.

### BRIEF DESCRIPTION OF THE DRAWINGS

FIG. 1 shows the effects of total body irradiation on the survival rate of mice in Example 1.

FIG. 2 shows the effects of abdominal irradiation on the survival rate of mice in Example 2.

FIG. 3 shows the effects of JAC4 on spleen and thymus indexes in mice receiving total body irradiation in Example 3.

FIG. 4 shows the HE staining images of spleen tissues of the mice in Example 3.

FIG. 5 shows the HE staining images of thymus tissues of the mice in Example 3.

FIG. 6 shows the blood biochemistry results after JAC4 treatment in mice receiving total body irradiation in Example 3.

FIG. 7 shows the effects of JAC4 on the expression level of JWA in small intestine epithelial cells of the mice in Example 4.

FIG. 8 shows the dose-response relationship of the protective effect of JAC4 on total body irradiation-induced injury in Example 5.

FIG. 9 shows the results of JAC4 treatment for preventing small intestine epithelial damage in the mice caused by total body irradiation in Example 5.

FIG. 10 shows the results of prophylactic JAC4 administration for alleviating small intestine epithelial damage in the mice caused by total body irradiation in Example 6.

FIG. 11 shows the results of therapeutic JAC4 administration for alleviating small intestine epithelial damage in the mice caused by total body irradiation in Example 7.

FIG. 12 shows the results of small intestine epithelial damage caused by abdominal irradiation in the mice in Example 8.

FIG. 13 shows the results of JAC4 alleviating body weight loss caused by abdominal irradiation in mice in Example 9.

FIG. 14 shows the results of JAC4 alleviating small intestine epithelial damage caused by abdominal irradiation in the mice in Example 10.

FIG. 15 shows the effects of JAC4 on the lengths of the small intestine and colon in mice after abdominal irradiation in Example 11.

FIG. 16 shows the effects of JAC4 on small intestine barrier damage, inflammatory and oxidative stress responses, apoptosis, and pathological structure of the small intestine caused by abdominal irradiation in wild-type mice and intestinal epithelial JWA-knockout mice in Example 12.

FIG. 17 shows the results of JAC4 reducing the release of cytochrome C in IEC-6 cells after radiation in Example 13.

FIG. 18 shows the results of JAC4 reducing the accumulation of reactive oxygen species and apoptosis in IEC-6 cells after radiation in Example 13.

FIG. 19 shows the results of JAC4 alleviating DNA double-strand damage in IEC-6 cells after radiation in Example 13.

FIG. 20 shows the results of JAC4 alleviating early apoptosis in IEC-6 cells caused by radiation in Example 13.

FIG. 21 shows the results of JAC4 alleviating apoptosis in IEC-6 cells in Example 14.

FIG. 22 shows the NMR spectrum of R-JAC4 in Example 15.

FIG. 23 shows the mass spectrum of R-JAC4 in Example 15.

FIG. 24 shows the plasma FD4 concentrations in mice in the control group and treatment groups in Example 18.

FIG. 25 shows the statistical results of small intestine length of the mice in the control group and treatment groups in Example 18.

FIG. 26 shows the plasma FD4 concentrations in the intestinal epithelial cell JWA-knockout mice (JWA^{IEC-KO}) and their wild type littermates (JWA^{IEC-WT}) in Example 19.

FIG. 27 shows the plasma levels of proinflammatory cytokines TNF-α and IL-1β in the intestinal epithelial cell JWA-knockout mice (JWA^{IEC-KO}) and their wild type littermates (JWA^{IEC-WT}) in Example 19.

FIG. 28 shows the synergistic effect of JAC4 on the X-ray radiation therapy in subcutaneous SPCA-1 xenograft tumor-bearing mouse model in Example 20.

FIG. 29 shows the results of JAC4 inhibiting radiation-induced inflammatory responses in mice in Example 21.

FIG. 30 shows the results of JAC4 inhibiting radiation-induced DNA damage in lung tissue of mice in Example 21.

FIG. 31 shows the effects of JAC4 in combination with radiation on SPCA-1 and BEAS-2B cells in Example 22.

FIG. 32 shows the effects of JAC4 in combination with radiation on DNA damage in SPCA-1 and BEAS-2B cells in Example 22.

FIG. 33 shows the effects of JAC4 in combination with radiation on apoptosis in SPCA-1 and BEAS-2B cells in Example 22.

FIG. 34 shows the results of JAC4 increasing the antioxidant capacity of BEAS-2B cells in Example 22.

FIG. 35 shows the results of JAC4 inhibiting the translocation of NF-κB to the nucleus in Example 22.

FIG. 36 shows the results of the JWA gene deletion reducing the synergistic and toxicity-alleviating effects of X-ray on SPCA-1 and BEAS-2B cells in Example 22.

FIG. 37 shows the effects of the JWA gene deletion on radiation-induced DNA damage and apoptosis in SPCA-1 and BEAS-2B cells in Example 22.

FIG. 38 shows the results of JAC4 alleviating radiation-induced damage to the integrity of single-cell-layer intestinal epithelium in Example 23.

### DETAILED DESCRIPTION

The present disclosure is further described below with reference to specific examples; the advantages and features of the present disclosure will become more apparent with the description. Experimental procedures without specified conditions in the examples are conducted according to conventional conditions or conditions recommended by the manufacturers. Reagents or instruments without specified manufacturers used herein are conventional products that are commercially available. Unless otherwise defined herein, scientific and technical terms used in correlation with the present disclosure shall have the meanings that are commonly understood by those skilled in the art.

The examples herein are illustrative only, and do not limit the scope of the present disclosure in any way. It will be appreciated by those skilled in the art that various modifications or substitutions may be made to the technical solutions of the present disclosure in form and details without departing from the spirit and scope of the present disclosure, and that these modifications and substitutions shall fall within the claimed scope of the present disclosure.

The C57BL/6 mice (25-30 g in body weight) aged 10 weeks used in the experiments of the present disclosure were SPF-grade animals from Shanghai SLAC Laboratory Animal Co., Ltd., China. Animal use was approved by the Institutional Animal Care and Use Committee of Nanjing Medical University (IACUC-2004044). X-ray irradiations were performed at the Nanjing Medical University Animal Center using an Rs-2000 Pro X-ray irradiator (RAD SOURCE, USA), with a dose rate of 1.25 Gy/min, and the abdominal irradiation covered an area of 3 cm in width above the iliac joints.

In the drawings of the present disclosure, * denotes P<0.05, ** denotes P<0.01, *** denotes P<0.001, **** denotes P<0.0001, ns or NS denotes no statistically significant difference, #denotes P<0.05,±#denotes P<0.01, ###denotes P<0.001, ####denotes P<0.0001, ! denotes P<0.05, and !! denotes P<0.01.

### Abbreviations

JAC4: compound of formula (I); R-JAC4: the R configuration of the compound of formula (I); S-JAC4: the S configuration of the compound of formula (I); ALT: alanine aminotransferase; AST: aspartate aminotransferase; TBI: total body irradiation; ABI: abdominal irradiation; BSA: bovine serum albumin; BER: base-excision repair; Bcl-2: B-cell lymphoma-2; Bax: Bcl-2 associated X gene; Caspase: cysteinyl aspartate-specific proteinase; CK-MB: creatine kinase isoenzymes MB; CAT: catalase; DAO: D-amino-acid oxidase; DAPI: 4′,6-diamidino-2-phenlindole; DCFH-DA: 2′,7′-dichlorodihydrofluorescein diacetate; DMSO: dimethyl sulfoxide; ELISA: enzyme linked immune sorbent assay; FITC-dextran: FITC-labeled dextran; GSH-PX: glutathione peroxidase; GSH: glutathione; LDH: lactic dehydrogenase; PAGE: polyacrylamide gel electrophoresis; PARP1: poly (ADR-ribose) polymerase-1; PBS: phosphate buffered solution; ROS: reactive oxygen species; SDS: sodium dodecyl sulfate; SOD: superoxide dismutase; TEMED: N,N,N′,N′-tetramethylethylenediamine; TNF: trinitrophenol, tumor necrosis factor; TBS: Tris-buffered saline; XRCC1: X-ray cross-complementing group 1; IEC: intestinal epithelial cell.

### Example 1. Effect of JAC4 on Survival Rate of Mice Receiving Total Body X-Ray Irradiation

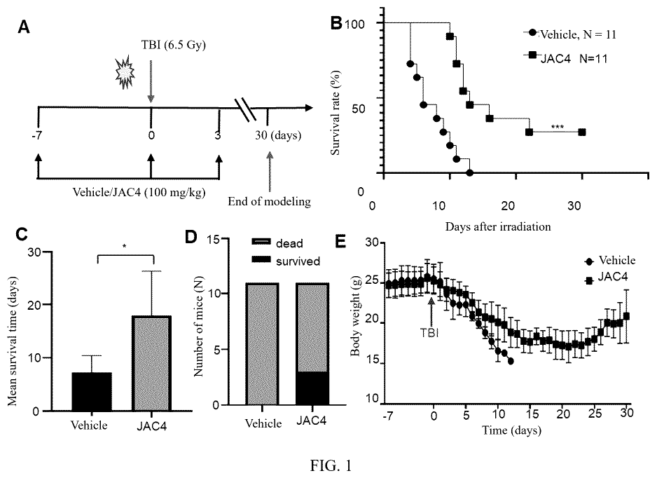

C57BL/6 male mice (24.8±1.6 g in body weight) aged 10 weeks were divided into two groups of 11 mice each. After acclimating to the environment, JAC4 (100 mg/kg) or an equal volume of vehicle (polyethylene glycol:ethanol:physiological saline=47.5:7.5:50, v/v/v) was orally administered to the mice once daily for 7 consecutive days. After that, the mice were subjected to total body X-ray irradiation (6.5 Gy) and JAC4 or vehicle treatment was continued. After 3 days, the administration of JAC4 or vehicle was discontinued. The status was observed daily and the body weights of the mice were recorded. The observation results on day 30 after radiation are shown in FIG. 1.

The results show that JAC4 significantly prolonged the survival of mice (p<0.05) (FIGS. 1B and 1C) and increased the survival rates of mice after radiation (FIG. 1D); the observation results on day 30 after radiation show that the mean survival after radiation was 7.2±3.1 days for mice in the vehicle control group, and 17.9±8.4 days for the JAC4 treatment group (FIG. 1C); also, the weight loss of the mice in the JAC4 treatment group after radiation was slower (FIG. 1E). Also, all mice in the vehicle control group after radiation treatment died on day 12, while 6 mice died and 5 mice survived in the JAC4 pretreatment group.

Therefore, the JAC4 treatment significantly prolonged the mean survival time of the mice after total body irradiation, increased the survival rate of the mice after radiation, and slowed the weight loss of the mice in the JAC4 treatment group.

### Example 2. Effect of JAC4 on Survival Rate of Mice after Abdominal X-Ray Irradiation

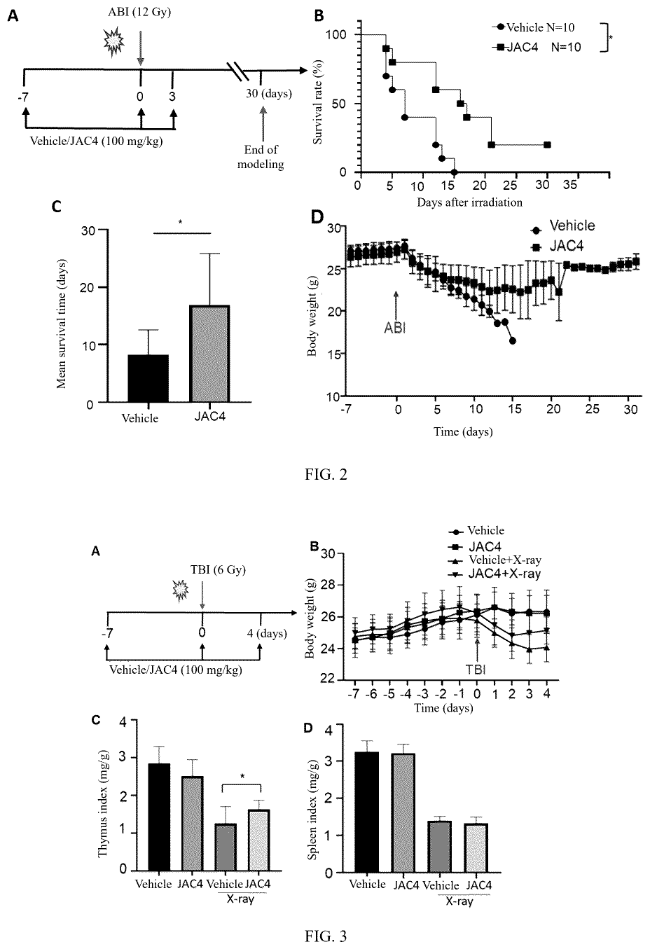

C57BL/6 male mice (26.7±1.0 g in body weight) aged 10 weeks were divided into two groups of 10 mice each. After acclimating to the environment, JAC4 (100 mg/kg) or an equal volume of vehicle (polyethylene glycol:ethanol:physiological saline=47.5:7.5:50, v/v/v) was orally administered to the mice once daily for 7 consecutive days. After that, the mice were subjected to abdominal X-ray irradiation (12 Gy) and JAC4 or vehicle treatment was continued. After 3 days, the administration of JAC4 or vehicle was discontinued. The status was observed daily and the body weights of the mice were recorded. The observation results on day 30 after radiation are shown in FIG. 2.

The results show that JAC4 significantly prolonged the survival of mice receiving abdominal X-ray irradiation (p<0.05) (FIG. 2C) and increased the survival rates of mice after radiation (FIG. 2B); the observation results on day 30 after radiation show that the mean survival was 8.3±4.2 days for mice in the vehicle control group, and 16.8±9.0 days for the JAC4 treatment group (FIG. 2C); also, the weight loss of the mice in the JAC4 treatment group after radiation was slower (FIG. 2D). After the radiation treatment, all mice in the vehicle control group died on day 15, while 4 mice died and 6 mice survived in the JAC4 pretreatment group at that time.

Therefore, the JAC4 treatment significantly prolonged the mean survival time of the mice after abdominal irradiation, increased the survival rate of the mice after radiation, and slowed the weight loss of the mice in the JAC4 treatment group.

### Example 3. Effect of JAC4 on Spleen and Thymus Indexes and Hematopoietic System in Mice after Total Body Irradiation

C57BL/6 male mice (24.8±1,6 g in body weight) aged 10 weeks were divided into 4 groups of 8 mice each. After acclimating to the environment, JAC4 (100 mg/kg) or an equal volume of vehicle (polyethylene glycol:ethanol:physiological saline=47.5:7.5:50, v/v/v) was orally administered to the mice once daily for 7 consecutive days. After that, the mice in one JAC4 treatment group and one vehicle group were subjected to total body X-ray irradiation (6 Gy) and JAC4 or vehicle treatment was continued. After 3 days, the administration of JAC4 or vehicle was discontinued, and blood samples, small intestine tissues, and thymus and spleen tissues were collected 4 days later. The results are shown in FIG. 3.

The results show that after X-ray radiation, the mice experienced a significant and continuous body weight loss from days 1-3; starting from day 4, the body weight of the mice was gradually stabilized. In the JAC4 treatment group, the mice experienced a significant and continuous body weight loss on days 1 and 2, and from day 3 onwards, the body weight started to rebound. The degree of body weight loss in mice in the JAC4 treatment group was significantly less than that in the control group (FIG. 3B).

The results show that in mice, the thymus index was 2.84±0.45 mg/g for the non-radiation vehicle control group, 2.51±0.43 mg/g for the non-radiation JAC4 treatment group, 1.25±0.46 mg/g for the vehicle+radiation group, and 1.63±0.24 mg/g for the JAC4+radiation group. The thymus index in mice subjected to X-ray radiation was significantly lower than that in the vehicle control group and JAC4 treatment group (p<0.05). Additionally, the thymus organ index in X-ray-irradiated mice treated with JAC4 showed a significant improvement, and was higher than that in the vehicle control group receiving X-ray radiation (p<0.05) (FIG. 3C).

The results show that in mice, the spleen index was 3.23±0.31 mg/g for the non-radiation vehicle control group, 3.22±0.26 mg/g for the non-radiation JAC4 treatment group, 1.39±0.12 mg/g for the vehicle+radiation group, and 1.31±0.18 mg/g for the JAC4+radiation group. The spleen index in mice subjected to X-ray radiation was significantly decreased (p<0.05), but JAC4 did not show improvement in the spleen index (FIG. 3D).

HE staining was performed on spleen tissues and thymus tissues from mice in all groups, with procedures as follows:

(1) Section deparaffinization: 1) xylene I treatment for 5 min; 2) xylene II treatment for 5 min; 3) xylene III treatment for 5 min; 4) 100% ethanol treatment for 5 min; 5) 100% ethanol II treatment for 5 min; 6) 95% ethanol treatment for 5 min; 7) 90% ethanol treatment for 5 min; 8) 85% ethanol treatment for 5 min; 9) 70% ethanol treatment for 5 min; 10) 60% ethanol treatment for 5 min.

(2) HE staining: After being rinsed with double-distilled water, the sections were stained with hematoxylin solution for 6 min, differentiated with 0.5% hydrochloric acid in alcohol for a short period, and then placed in double-distilled water for bluing. When the chromatin within the cell nuclei was clearly visible under a microscope, the sections were stained with 0.5% eosin for 30 s.

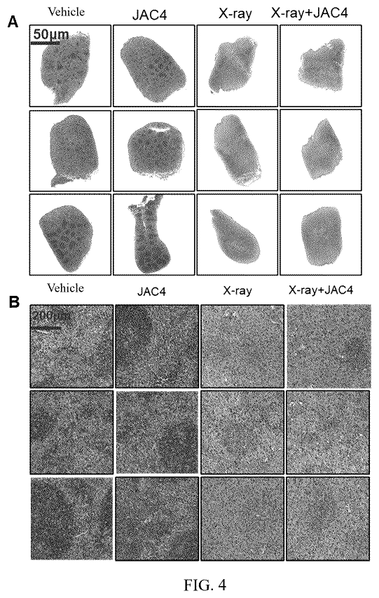

The HE staining images of spleen tissues of the mice are shown in FIG. 4. The spleen structures of the mice in the vehicle control group and the mice treated with JAC4 only without X-ray radiation were intact, with clear boundaries between the red pulp and white pulp, distinct germinal centers and marginal zones, and no abnormalities. After X-ray radiation, the spleens of mice became smaller in volume, with shriveled capsules, and the boundaries between the red pulp and white pulp were unclear. The white pulp was significantly decreased, and the red pulp was decreased. There was no significant difference between the vehicle control group and the JAC4 group (FIGS. 4A and 4B).

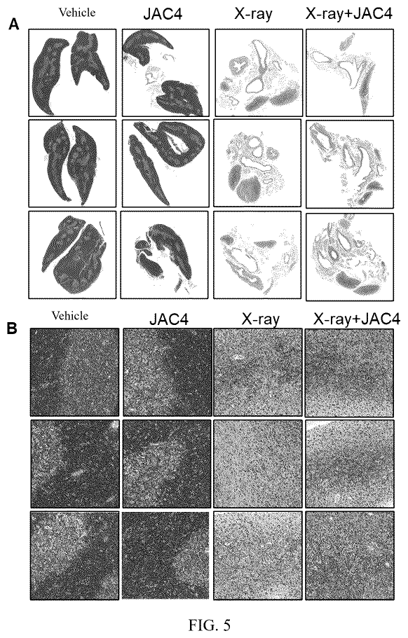

The HE staining images of mouse thymus tissues are shown in FIG. 5. The thymus tissues of the mice in the vehicle control group and the mice treated with JAC4 only without X-ray radiation exhibited clear lobular structures with distinct corticomedullary boundaries. After X-ray radiation, cavities were observed in the tissues, and the corticomedullary boundaries were unclear and unrecognizable. However, there was no significant difference between the vehicle control group and the JAC4 group (FIGs. 5A and 5B).

The biochemical results of the mice were tested, with procedures as follows: Peripheral blood was collected from the orbit into an ethylenediaminetetraacetic acid K3 tube, followed by immediate blood biochemistry analysis on a blood analyzer.

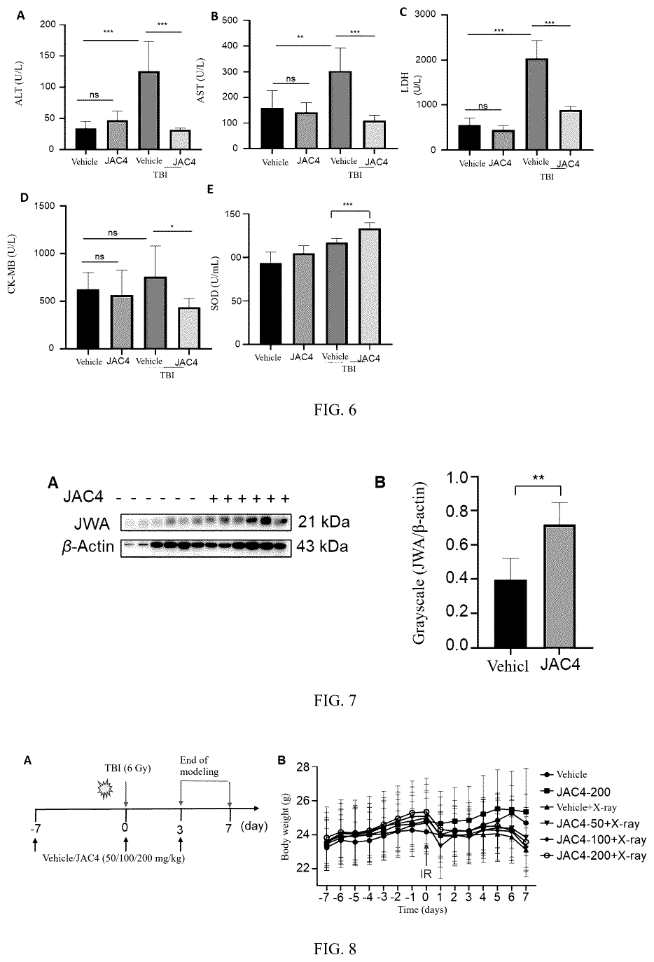

The biochemical results of the mouse blood are shown in FIG. 6: ALT (the non-radiation vehicle control group: 33.83±11.18 U/L, the non-radiation JAC4 treatment group: 47.17±14.81 U/L, the vehicle+radiation group: 126.17±14.81 U/L, and the JAC4+radiation group: 31.33±3.14 U/L), AST (the non-radiation vehicle control group: 158.83±38.60 U/L, the non-radiation JAC4 treatment group: 140.83±38.72 U/L, the vehicle+radiation group: 303.83±89.06 U/L, and the JAC4+radiation group: 110.17±20.86 U/L), LDH (the non-radiation vehicle control group: 554.33±156.63 U/L, the non-radiation JAC4 treatment group: 444.00±94.58 U/L, the vehicle+radiation group: 2037.67±398.94 U/L, and the JAC4+radiation group: 888.67±81.48 U/L), CK-MB (the non-radiation vehicle control group: 626.00±173.22 U/L, the non-radiation JAC4 treatment group: 565.50±261.37 U/L, the vehicle+radiation group: 759.67±319.62 U/L, and the JAC4+radiation group: 437.67±90.38 U/L), and SOD (the non-radiation vehicle control group: 93.50±12.55 U/mL, the non-radiation JAC4 treatment group: 104.50=9.09 U/mL, the vehicle+radiation group: 117.17±4.67 U/mL, and the JAC4+radiation group: 133.00±6.96 U/mL). The above results show that after total body X-ray irradiation, liver damage indicators, such as ALT and AST, increased (p<0.001) (FIGS. 6A and 6B), and cardiac damage indicators, such as LDH, CK-MB, and superoxide dismutase SOD, also increased (p<0.001) (FIGS. 6C-6E), while treatment with JAC4 ameliorated the above phenomena.

### Example 4. Effect of JAC4 on Small Intestine Mucosa Epithelial Damage Caused by X-Ray Radiation

C57BL/6 male mice (24.8±1.6 g in body weight) aged 10 weeks were divided into 2 groups. After acclimating to the environment, JAC4 (100 mg/kg) or an equal volume of vehicle (polyethylene glycol:ethanol:physiological saline=47.5:7.5:50, v/v/v) was orally administered to the mice once daily for 7 consecutive days. After that, the mice were subjected to total body X-ray irradiation (6 Gy) and JAC4 or vehicle treatment was continued for 4 days, after which small intestine tissues were collected. The expression levels of JWA in the small intestine were detected by Western blot, with β-actin as the reference. Adobe photoshop CC was used to measure the grayscale value of JWA/β-actin in panel A for statistical analysis (** p<0.01). The results are shown in FIG. 7.

The Western blot procedures are as follows:

1. Protein sample preparation: Proper volumes of protein sample solutions were mixed with physiological saline to give 40 μg/μL solutions at a volume of 16 μL, and 4 μL of 5×SDS sample buffer was added. The mixture was uniformly mixed, centrifuged at 1000×g for 10 s, and boiled at 100° C. for 5 min. The samples were cooled at room temperature and then preserved in a −20° C. freezer.

2. SDS-PAGE preparation:

(1) Lower separating gel: The glass plates and gel cassette were assembled while ensuring that the bottom of the glass plates was sealed to prevent leakage of the gel. Based on the molecular weight of the proteins of interest, separating gels with different formulation ratios were prepared. To a 50 mL tube, double-distilled water, separating gel buffer, 30% polyacrylamide solution, 10% SDS, AP, and TEMED were sequentially added in corresponding proportions. The mixture was uniformly mixed and immediately poured into the glass plates, followed by sealing with absolute ethanol. A distinct boundary observed between the separating gel layer and the liquid sealing layer indicated that the polymerization of the separating gel was completed, taking about 1 h.

(2) Upper stacking gel: The absolute ethanol was discarded, and the gel was rinsed with double-distilled water 2-3 times. Then the residual liquid on the surface was removed with absorbent paper. To a 50 mL tube, 2 mL of double-distilled water, 1 mL of stacking gel buffer, 1 mL of 30% polyacrylamide solution, 40 L of 10% SDS, 20 L of 10% AP, and 8 L of TEMED were sequentially added. The mixture was uniformly mixed and then poured into the headspace above the prepared separating gel while taking care to avoid the formation of air bubbles. The sample comb was slowly inserted. The polymerization was performed at room temperature for about 30 min.

(3) The glass plates were removed from the mold. The gels were placed into the electrophoresis tank, and the pre-prepared electrophoresis buffer was added. Then the sample comb was slowly removed.

3. Protein loading: After the protein samples were thawed on ice and mixed by vortexing, a total volume of 20 μL of sample or 5 L of protein molecular weight markers was added to the lanes.

4. Electrophoresis: The electrophoresis buffer was topped up, the power supply was connected, and the electrophoresis system was turned on to perform the electrophoresis. Stacking gel electrophoresis was performed at 60 V for about 40 min, and separating gel electrophoresis was performed at 90 V for about 2-3 h.

5. Gel cutting: The gel between the two glass plates was scraped off using a gel-cutting plate. After the upper stacking gel was removed, the remaining gel was soaked in a transfer buffer.

6. Transfer: A PVDF membrane with an area slightly larger than the gel block was cut and marked. The membrane was activated in methanol for 30 s and then placed in 1× transfer buffer to keep it moist. The transfer “sandwich” was assembled in sequence, with the gel on the black side (cathode) and the PVDF membrane on the red side (anode). During the assembly process, air bubbles were avoided and the “sandwich” was compressed tightly to ensure the transfer efficiency. The “sandwich” was inserted into the transfer electrophoresis tank, and the tank was filled with 1×transfer buffer. The leads from the electrophoresis tank were connected to the corresponding connectors on the electrophoresis system. The system was turned on, the current and time were adjusted, and the transfer was initiated. Due to the heat generated during the transfer process, the electrophoresis tank was placed in an ice bath throughout the procedure. For target proteins with a molecular weight greater than 100 kDa, the transfer was performed at a constant current of 200 mA for 3 h; for target proteins with a molecular weight less than 100 kDa, the transfer was performed at a constant current of 220 mA for 1.5 h.

7. Blocking: The PVDF membrane was placed in an incubation box of a suitable size with the protein side facing upward, 5% skim milk or 5% BSA was added to cover the PVDF membrane, followed by incubation at room temperature for 1 h for blocking. The blocking buffer was discarded, and the PVDF membrane was rinsed in 1×TBST for 5 min.

8. Co-incubation with primary antibody: The PVDF membrane was cut according to the molecular weight of the target protein. The primary antibodies were diluted and added as per the instructions of the antibodies (internal control tubulin: Beyotime, 1:1000, Cat. No.: AT819, mouse antibody; EGFR: CST, 1:1000, Cat. No.: 4267S, rabbit antibody), and incubated with the PVDF membrane on a shaker at 4° C. overnight.

9. Membrane washing: The PVDF membrane was taken out from the primary antibody dilutions and washed with 1×TBST thrice on a shaker at room temperature for 10 min.

10. Co-incubation with secondary antibody: The PVDF membrane was incubated with the secondary antibody diluted as per the instructions of the antibody at 37° C. for 1 h.

11. Membrane washing: The PVDF membrane was taken out from the secondary antibody dilutions and washed with 1×TBST 4 times on a shaker at room temperature for 15 min.

12. Chemiluminescence: 200 μL of chemiluminescent solution, prepared as per the kit instructions, was uniformly applied to each PVDF membrane. The membranes were then developed and photographed using a chemiluminescence gel imaging system, and the images were saved.

13. Result processing: The Western blot bands obtained from the experiment were measured for their grayscale values using the Adobe photoshop CC software, followed by semi-quantitative analysis. The results were then normalized using the corresponding internal control band.

As can be seen from FIG. 7, the mean grayscale value of JWA expression after JAC4 treatment was 0.72±0.13, while the mean grayscale value of JWA expression in the vehicle control group was 0.40±0.12, indicating that JAC4 treatment can significantly increase the expression of JWA in the small intestine epithelial tissue of mice.

### Example 5. Dose-Response Relationship of the Protective Effect of JAC4 on Total Body X-Ray Radiation-Induced Injury

C57BL/6 male mice (24.8±1.6 g in body weight) aged 10 weeks were divided into 6 groups: a vehicle control group, a JAC4 (200 mg/kg) treatment group, a vehicle control+radiation group, and JAC4 intervention (50 mg/kg, 100 mg/kg, and 200 mg/kg)+radiation groups, with 12 mice in each group. JAC4 or an equal volume of vehicle (polyethylene glycol:ethanol:physiological saline=47.5:7.5:50, v/v/v) was orally administered to the mice once daily for 7 consecutive days. After that, the mice in the radiation groups were subjected to total body X-ray irradiation (6 Gy). The JAC4 or vehicle treatment was continued for 3 days.

The body weights of the mice before and after the total body X-ray irradiation were measured, and the body weight curves are shown in FIG. 8. As can be seen from FIG. 8, after X-ray radiation, the mice experienced a rapid body weight loss, which then plateaued, but continued on day 6. The mice in the JAC4 treatment groups consistently showed a slower body weight loss compared with the vehicle+radiation group.

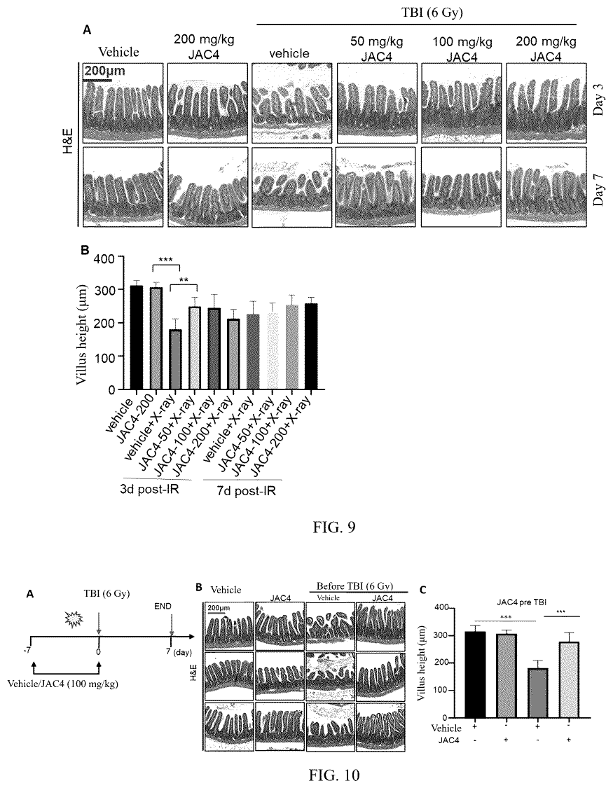

HE staining was performed on the small intestine tissues of mice subjected to total body X-ray irradiation. The length of the small intestine villi in all groups was measured and the results were statistically analyzed. The specific procedures are as follows: 3 mice were randomly selected from each group for statistical analysis of their small intestine tissue HE staining images, and for each mouse, 6 fields of view were randomly selected for inspection (** p<0.01, *** p<0.001). The results are shown in FIG. 9.

As can be seen from FIG. 9, the measurement results show that the small intestine villus length was 312.28±14.64 μm for the non-radiation vehicle control group and 306.78±14.64 μm for the non-radiation JAC4 treatment (200 mg/kg) group (p>0.05), indicating that the JAC4 monotherapy had no significant effect on the length of the small intestine villi. On day 3 after X-ray radiation, the measurement results show that the small intestine villus length was 180.91±31.42 μm for the vehicle group, and 249.42±27.19 μm, 244.78±40.74 μm, and 212.43±27.99 μm for the 3 treatment groups treated with JAC4, respectively. Compared with the non-radiation group, the small intestine epithelium of the mice after radiation exhibited significant damage, characterized by a reduction in villus number, breaks, and reduced length (p<0.05). Compared with the vehicle+radiation group, the damage to the small intestine epithelial villi was ameliorated in the 3 treatment groups after JAC4 intervention (p<0.05); the trend indicates that the 50 mg/kg group had a better effect than those of the 100 mg/kg and 200 mg/kg groups. On day 7 after radiation, the measurement results show that the small intestine villus length was 225.93±39.31 μm for the vehicle+radiation group and 235.57±24.34 μm, 254.95±28.91 μm, and 259.17±17.56 μm for the 3 treatment groups receiving JAC4, respectively. It can be seen that on day 7, the small intestine epithelial stem cells of the mice were renewed and the epithelium was gradually repaired. Compared with the vehicle+radiation group, the small intestine epithelial villi of the JAC4+radiation groups were longer; as can be seen from the trend of the three treatment+radiation groups, JAC4 intervention exhibited a promoting effect on the repair of small intestine villi on day 7 with a certain dose-response relationship.

### Example 6. Protective Effect of Prophylactic JAC4 Administration on X-Ray Radiation-Induced Injury

C57BL/6 male mice (24.8±1.6 g in body weight) aged 10 weeks were divided into 4 groups. JAC4 (100 mg/kg) or an equal volume of vehicle (polyethylene glycol:ethanol:physiological saline=47.5:7.5:50, v/v/v) was orally administered to the mice once daily for 7 consecutive days. After that, the mice were subjected to total body X-ray irradiation (6 Gy) and JAC4 or vehicle treatment was discontinued. After 7 days, the small intestine tissues were collected for HE staining and inspection, and the small intestine villus length was measured in all groups and statistically analyzed. The specific procedures are as follows: 3 mice were randomly selected from each group for statistical analysis of their small intestine tissue HE staining images, and for each mouse, 6 fields of view were randomly selected for inspection (*** p<0.001). The results are shown in FIG. 10.

As can be seen from FIG. 10, the results show that the small intestine villus length was 314.74±22.90 μm for the non-radiation vehicle group, 306.54±14.00 μm for the non-radiation JAC4 treatment group, 181.85±14.00 μm for the vehicle+radiation group, and 277.64±33.55 μm for the JAC4 pretreatment+radiation group. Compared with the vehicle+radiation group, JAC4 prophylactic administration significantly ameliorated the reduction in villus number, breaks, and the reduction in small intestine villus length (p<0.05).

### Example 7. Protective Effect of Therapeutic JAC4 Administration on X-Ray Radiation-Induced Injury

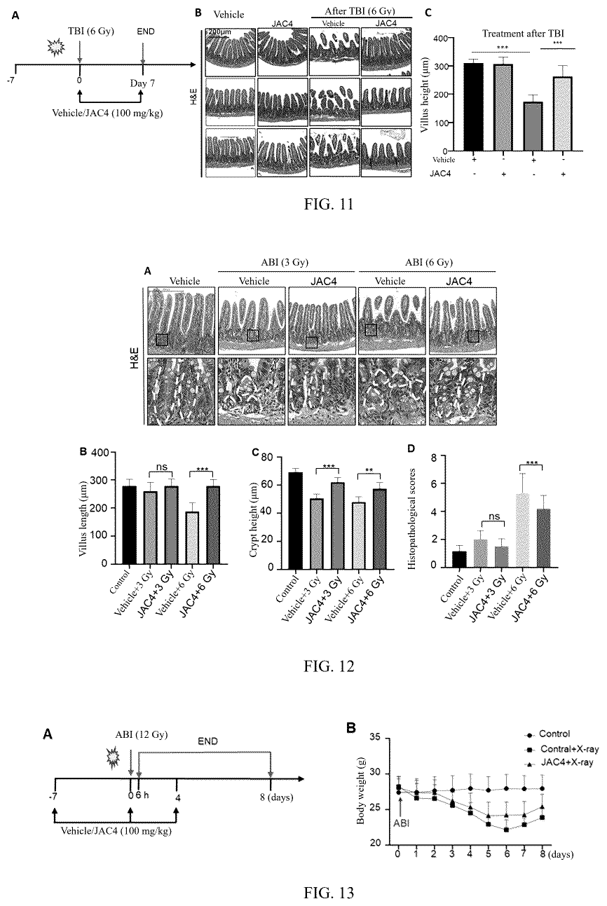

C57BL/6 male mice (24.8±1.6 g in body weight) aged 10 weeks were divided into 4 groups. The mice were subjected to total body X-ray irradiation (6 Gy) before JAC4 (100 mg/kg) or an equal volume of vehicle (polyethylene glycol:ethanol:physiological saline=47.5:7.5:50, v/v/v) was orally administered to the mice once daily for 7 consecutive days. The small intestine tissues were collected for HE staining and inspection, and the small intestine villus length was measured in all groups and statistically analyzed. The specific procedures are as follows: 3 mice were randomly selected from each group for statistical analysis of their small intestine tissue HE staining images, and for each mouse, 6 fields of view were randomly selected for inspection (*** p<0.001). The results are shown in FIG. 11.

As can be seen from FIG. 11, the results show that the small intestine villus length was 310.86±13.14 μm for the non-radiation vehicle group, 306.71±24.22 μm for the non-radiation JAC4 treatment group, 173.16±24.22 μm for the radiation+vehicle group, and 263.11±37.99 μm for the radiation+JAC4 treatment group. Compared with the vehicle+radiation group, the JAC4 treatment after X-ray radiation can also ameliorate the reduction in villus number, breaks, and the reduction in small intestine villus length (p<0.05).

### Example 8. Small Intestine Epithelial Injury in Mice Caused by Different Doses of X-Ray Radiation

CS7BL/6 male mice (24.8±1.6 g in body weight) aged 10 weeks were divided into 5 groups of 6 mice each. JAC4 (100 mg/kg) or an equal volume of vehicle (polyethylene glycol:ethanol:physiological saline=47.5:7.5:50, v/v/v) was orally administered to the mice once daily for 7 consecutive days. After that, the mice were subjected to abdominal X-ray irradiation at different doses (3 or 6 Gy) and JAC4 or vehicle treatment was continued. After 4 days, the small intestine tissues were collected for HE staining and inspection, the small intestine villus length and the ileum crypt height in all groups were measured and statistically analyzed, and the small intestine was histopathologically scored. The specific procedures are as follows: 3 mice were randomly selected from each group for statistical analysis of their small intestine tissue HE staining images, and for each mouse, 6 fields of view were randomly selected for inspection (*** p<0.001). The results are shown in FIG. 12.

As can be seen from FIG. 12, the small intestine villus length was 278.62±24.15 μm for the non-radiation vehicle group, 259.68±32.61 μm for the vehicle+3 Gy group, and 278.19±25.21 μm for the JAC4+3 Gy group (p>0.05); and 187.11±31.70 μm for the vehicle+6 Gy group and 278.25±23.79 μm for the JAC4+6 Gy group (p<0.05). The small intestine histopathological score was 1.17±0.41 for the non-radiation control group, 2.00±0.63 for the vehicle+3 Gy group, and 1.50±0.55 for the JAC4±3 Gy group (p>0.05); and 5.33±1.37 for the vehicle+6 Gy group and 4.17±0.98 for the JAC4±6 Gy group (p<0.05). The ileum crypt height was 69.12±2.81 μm for the non-radiation control group, 50.40+3.26 μm for the vehicle+3 Gy group, and 62.06±3.26 μm for the JAC4+3 Gy group (p<0.05); and 47.86±3.75 μm for the vehicle+6 Gy group and 57.32±4.43 μm for the JAC4+6 Gy group (p<0.05). The above results indicate that for the vehicle group, X-ray radiation at 3 Gy resulted in mild damage to the small intestine epithelium, with no significant changes observed within 4 days. After X-ray radiation at 6 Gy, there were a shortening of the small intestine villi, a decrease in crypt height, and an increase in histopathological scores. The JAC4 treatment group exhibited amelioration in all of the above damages.

### Example 9. Alleviation of Abdominal Irradiation-Induced Body Weight Loss in Mice by JAC4

CS7BL/6 male mice aged 10 weeks were divided into 3 groups, i.e., a vehicle control group, a vehicle+radiation group, and a JAC4+radiation group 8 days after radiation, with 6 mice in each group. JAC4 (100 mg/kg) or an equal volume of vehicle (polyethylene glycol:ethanol:physiological saline=47.5:7.5:50, v/v/v) was orally administered to the mice once daily for 7 days. After that, the mice were subjected to abdominal X-ray irradiation (12 Gy) and the JAC4 or vehicle treatment was continued for 4 days. The body weights of the mice before and after total body X-ray irradiation were recorded daily, and body weight curves were plotted. The results are shown in FIG. 13.

As can be seen from the mouse body weight curves in FIG. 13B, after abdominal X-ray irradiation, the mice experienced a rapid body weight loss, and from day 7 onwards, the body weight started to rebound. The body weight curve of the JAC4 treatment group was consistently above that of the vehicle+radiation group.

### Example 10. Alleviation of Abdominal Irradiation-Induced Small Intestine Epithelial Injury in Mice by JAC4

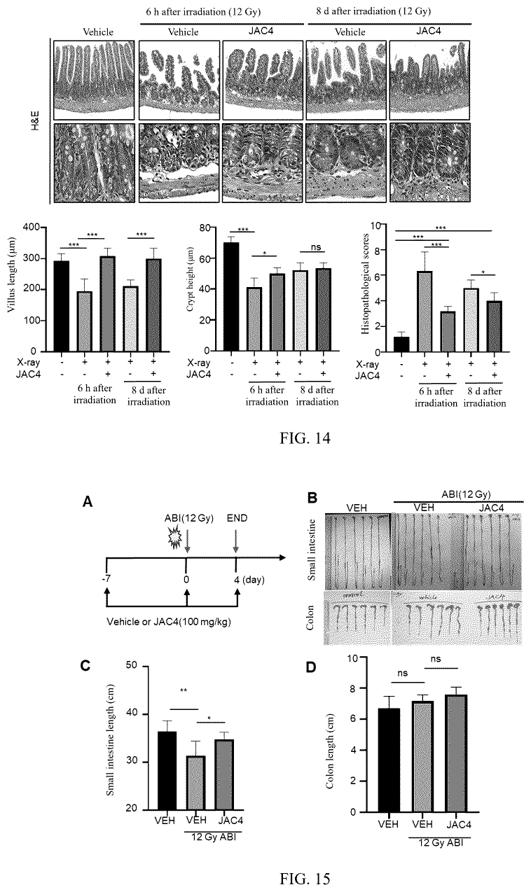

C57BL/6 male mice (24.7±0.9 g in body weight) aged 10 weeks were divided into 3 groups: a vehicle control group (n=6), a vehicle+radiation group (n=12), and a JAC4+radiation group (n=12). JAC4 (100 mg/kg) or an equal volume of vehicle (polyethylene glycol:ethanol:physiological saline=47.5:7.5:50, v/v/v) was orally administered to the mice once daily for 7 consecutive days. After that, the mice in the radiation groups were subjected to abdominal X-ray irradiation (12 Gy). The mice in the vehicle control group (n=6), the vehicle+radiation group (n=6), and the JAC4+radiation group (n=6) were sacrificed at 6 h, and blood samples and small intestine tissues were collected. The remaining mice in the vehicle+radiation group (n=6) and the JAC4+radiation group (n=6) were sacrificed after day 8, and blood samples and small intestine tissues were collected. In the process, abdominal irradiation was given while sparing the femoral region to minimize damage to the hematopoietic system. After the abdominal irradiation, HE staining was performed on the small intestines of mice in all groups, and the small intestine villus lengths, histopathological scores of the small intestine tissue sections, and the crypt heights were measured in all groups (* p<0.05, *** p<0.001). The results are shown in FIG. 14.

As can be seen in FIG. 14, ionizing irradiation led to a shortening of the small intestine mucosal villi and an increase in pathological scores, while JAC4 treatment can prevent the damage to the histological structure of the small intestine mucosa caused by radiation. The small intestine villus length was 293.54±22.21 μm for the non-irradiation control group. At 6 h after radiation, the small intestine villus length was 194.97±39.39 μm for the vehicle control group, while the small intestine villus length was 308.25±25.65 μm for the JAC4 treatment group (p<0.05); on day 8 after radiation, the small intestine villus length was 210.57±20.42 μm for the vehicle control group, while the small intestine villus length was 300.38±33.27 μm for the JAC4 treatment group (p<0.05). The small intestine histopathological score was 1.17±0.41 for the non-radiation control group. At 6 h after radiation, the small intestine histopathological score was 6.33±1.51 for the vehicle control group, while the small intestine histopathological score was 3.17±0.41 for the JAC4 treatment group (p<0.05); on day 8 after radiation, the small intestine histopathological score was 5.00±0.63 for the vehicle control group, while the small intestine histopathological score was 2.83±0.41 for the JAC4 pretreatment group (p<0.05). The small intestine crypt height was 70.42±3.52 μm for the non-radiation control group. At 6 h after radiation, the small intestine crypt height was 41.17±5.89 μm for the vehicle control group, while the small intestine crypt height was 49.80±4.06 μm for the JAC4 pretreatment group (p>0.05). On day 8 after radiation, the small intestine crypt height was 52.26±4.88 μm for the vehicle control group, while the small intestine crypt height was 53.76±3.33 μm for the JAC4 pretreatment group (p>0.05). The results show that the abdominal X-ray irradiation led to a shortening of the small intestine mucosal villi and an increase in pathological scores, while JAC4 treatment can prevent the damage to the histological structure of the small intestine mucosa caused by irradiation and promote the repair of the small intestine mucosa.

### Example 11. Alleviation of X-Ray-Induced Acute Radiation Enteritis by JAC4

C57BL/6 male mice (27.6±1.9 g in body weight) aged 10 weeks were divided into 3 groups of 6 mice each. JAC4 (100 mg/kg) or an equal volume of vehicle (polyethylene glycol:ethanol:physiological saline=40:7.5:52.5, v/v/v) was orally administered to the mice once daily for 7 consecutive days. After that, the mice were subjected to abdominal X-ray irradiation (12 Gy) and JAC4 or vehicle treatment was continued. After 4 days, blood samples and small intestine were collected.

The small intestine epithelial mucosal barrier is located at the interface between luminal micro-organisms and the mucosal immune system, and can maintain the intestinal mucosal barrier homeostasis. The integrity of the small intestine mucosal barrier is determined by a number of factors, including the tight junction proteins, proteins in other cell junction complexes, and the like. JAC4 has a protective effect on the small intestine mucosal barrier. The disruption of the small intestine mucosal barrier may lead to radiation enteritis and a reduction in the length of small intestine due to the inflammatory edema. The measured lengths of the small intestine and colon in mice are shown in FIG. 15.

As can be seen from FIG. 15, the small intestine length was 36.42±2.22 cm for the non-radiation vehicle group, 31.42±3.02 cm for the vehicle+radiation group, and 34.83±1.44 cm for the JAC4+radiation group. Compared with the non-radiation vehicle group, the length of the small intestine was shortened after X-ray radiation, while the small intestine length of the mice in the JAC4+radiation group was greater than that in the vehicle+radiation group (p<0.05) (FIGS. 15B and 15C); there were no statistically significant differences in the length of the colon among the groups (FIG. 15D) (* p<0.05, ** p<0.01, and ns: p>0.05).

### Example 12. Protection Against X-Ray-Induced Intestinal Injury in Mice by JAC4

The Model Animal Research Institute of Nanjing University was commissioned to conduct the construction of JWA^{flox/+} A129 mice and embryo cryopreservation, preservation, and thawing. These mice were bred with wild-type C57/BL6 mice for over six generations to give mice of C57/BL6 background. Villin-cre mice, provided by Shanghai Model Organisms Center, Inc., were C57/BL6 mice. The JWA-knockout mice (JWA^{ko}) and their littermate control (JWA^{wt}) counterparts were derived from an in-house conservation breed. The JWA^{flox/flox} mice were bred from the JWA^{flox/+} mice. The intestinal epithelial JWA-knockout mice (JWA^{IEC-ko}) and control group (JWA^{IEC-wt}) were obtained through breeding JWA^{flex/flex} mice with Villin-cre mice.

The off-target model was established using JWA^{IEC-ko} mice and their JWA^{IEC-wt} littermates (8 weeks old), and the mice receiving radiation were divided into 4 groups: (1) a JWA^{IEC-wt} mice+vehicle group (n=5); (2) a JWA^{IEC-wt} mice+JAC4 group (n=5); (3) a JWA^{IEC-ko} mice+vehicle group (n=6); (4) a JWA^{IEC-ko} mice+JAC4 group (n=5). All mice were subjected to abdominal irradiation at 10 Gy. 7 days before radiation, 10 mg/kg JAC4 or an equal volume of vehicle (polyethylene glycol:ethanol:physiological saline=40:7.5:52.5, v/v/v) was administered intragastrically daily until the end of modeling. The blank control group (wild type, n=6) was not irradiated, and the modeling was terminated on day 4 after radiation.

To verify whether the protective effect of JAC4 on X-ray radiation-induced intestinal damage in mice is dependent on the expression of JWA, the present disclosure verified the targeting effect of JAC4 using JWA^{IEC-ko} mice.

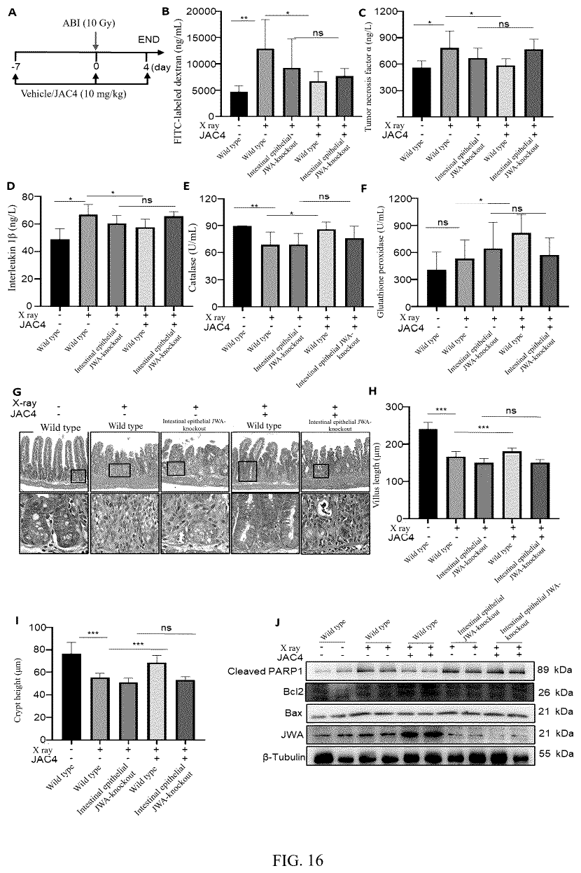

JWA^{IEC-ko} and JWA^{IEC-wt} mice were treated daily with JAC4 (10 mg/kg) or an equal volume of vehicle (polyethylene glycol:ethanol:physiological saline=47.5:7.5:50, v/v/v), respectively, from 7 days before the ABI (10 Gy) exposure to 4 days after the exposure. The detection of plasma biomarkers, including intestinal barrier (FD4), inflammation (TNF-α and IL-1β), and oxidative stress (CAT and GSH-px) indicators, shows that compared with the blank control group (4758.4±1066.0 ng/mL), the FD4 content in the vehicle group (12865.5±5539.5 ng/mL) significantly increased after radiation, whereas the FD4 content was significantly reduced (6719.9±1823.5 ng/mL) after treatment with JAC4. The results suggest that 10 mg/kg JAC4 can alleviate radiation-induced intestinal damage in wild-type mice. In JWA^{IEC-ko} mice, there was no significant difference in FD4 content after JAC4 treatment (9192.8±5573.2 vs. 7673.7±1474.2 ng/mL), indicating that JAC4 has no protective effects on the radiation-induced intestinal damage in JWA^{IEC-ko} mice (FIG. 16B). Meanwhile, the contents of TNF-α and IL-1β were consistent with the results of FD4. 10 mg/kg JAC4 reduced the inflammatory response in JWA^{IEC-wt} mice after radiation (TNF-α: 583.2±78.2 vs. 783.5±192.6 ng/mL; IL-1p: 57.76±5.9 vs. 66.7±7.5 ng/mL), but exhibited no similar effects in JWA^{IEC-ko} mice (TNF-α: 769.8±112.3 vs. 668.8±112.7 ng/mL; IL-1β: 65.6±3.3 and 66.7±7.5 ng/mL) (FIGS. 16C and 16D). As shown in panels E and F, ABI increased the activity of GSH-Px (530.8±207.8 vs. 406.2±197.9 U/mL) and decreased the activity of CAT (89.4 f 0.4 vs. 68.7±14.2 U/mL). JAC4 further enhanced the antioxidant enzymes in JWA^{IEC-wt} mice (815.1±203.9 vs. 530.8±207.8 U/mL and 85.9±8.2 vs. 68.7±14.2 U/mL; P<0.05, P<0.05). However, these results were not confirmed in JWA^{IEC-ko} mice. Histological analysis confirmed that in wild-type mice, the villus length and the crypt height were significantly reduced on day 4 after radiation in the vehicle group (villus: 166.3±14.4 vs. 240.7±17.4 μm; crypts: 55.3±3.7 vs. 76.7±10.0 μm). However, in JWA^{IEC-ko} mice, JAC4 did not ameliorate intestinal tissue damage (intestinal villus: 150.6±8.4 vs. 150.0±11.8 μm; crypt: 53.0±3.0 vs. 51.0±3.8 μm) (FIGS. 16G-16I). Intestinal tissues were isolated, and the expression of apoptotic molecules was detected by Western blot (FIG. 16J). In wild-type mice, compared with the control group, X-rays increased the expression of Bax and cleaved PARP1 in the small intestine. In contrast, the JAC4 treatment downregulated the expression of Bax and cleaved PARP1 in mice, but in JWA-^{IEC-ko} mice, the upregulation of anti-apoptotic molecule Bcl2 was not observed.

The above results indicate that JAC4 protects the intestinal epithelium in mice from X-ray radiation-induced damage by activating JWA; in JWA^{IEC-ko} mice, JAC4 cannot alleviate the damage to the irradiated intestinal epithelium by targeting JWA activation. Therefore, JAC4 is dependent on intestinal epithelial cell JWA expression to protect mice from X-ray-induced intestinal damage.

### Example 13. Effect of JAC4 on IEC-6 Cells

The effect was explored in a small intestinal epithelial IEC-6 cell in-vitro radiation model.

Formulation of JAC4 (1 μM or 10 μM): 5 mg of JAC4 was added to 1527 μL of DMSO to prepare a 10 mM stock solution, which was then serially diluted into 2 mM and 200 μM stock solutions.

10 μL of 2 mM stock solution was added into 90 μL of complete culture medium. The 100-μL mixture was uniformly mixed before 900 μL of complete culture medium was added, and uniformly mixed again before 1 mL of complete culture medium was added. The mixture was again uniformly mixed and then added into a six-well plate to give 10 μM JAC4.

10 μL of 200 μM stock solution was added into 90 μL of complete culture medium. The 100-μL mixture was uniformly mixed before 900 μL of complete culture medium was added, and uniformly mixed again before 1 mL of complete culture medium was added. The mixture was again uniformly mixed and then added into a six-well plate to give 1 μM JAC4.

Control wells: 10 μL of DMSO was added into 90 μL of complete culture medium. The 100-μL mixture was uniformly mixed before 900 μL of complete culture medium was added, and uniformly mixed again before 1 mL of complete culture medium was added. The mixture was again uniformly mixed and then added into a six-well plate.

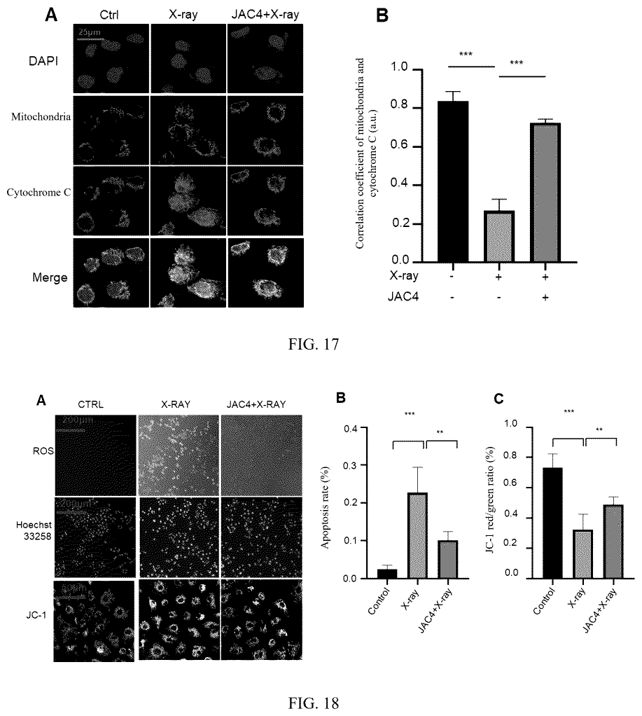

IEC-6 cells were pretreated for 24 h with 1 μM JAC4 or DMSO and subjected to radiation (12 Gy) treatment. After 24 h, colocalized cell immunofluorescent images of mitochondrial cytochrome C staining and mitochondria staining were acquired and subjected to fluorescent colocalization analysis using the Image J software to give colocalization correlation coefficients (***p<0.001) of cytochrome C and mitochondria in IEC-6 cells after different treatments. The results are shown in FIG. 17.

As can be seen from FIG. 17, the fluorescent colocalization correlation coefficient of the mitochondria and cytochrome C was 0.84+0.05 for the non-radiation control group, 0.27±0.06 for the vehicle+radiation group, and 0.73±0.02 for the JAC4+radiation group. The results show that after the IEC-6 cells were treated by X-ray radiation, the release of cytochrome C from mitochondria was significantly increased. A large amount of cytochrome C was released from mitochondria, and the colocalization coefficient of mitochondria and cytochrome C was reduced. JAC4 can prevent the massive release of cytochrome C.

IEC-6 cells were pretreated for 24 h with JAC4 (1 M) or DMSO and subjected to radiation (12 Gy) treatment. After 24 h the ROS produced in the IEC-6 cells was determined with a DCFH probe, the apoptosis of IEC-6 cells was determined by Hoechst staining, the mitochondrial membrane potential in IEC-6 cells was determined by JC-1 staining, and the apoptotic IEC-6 cell ratio of cells different treatments and the red/green fluorescence ratio in IEC-6 cells after different treatments were calculated (**p<0.01 and ***p<0.001). The results are shown in FIG. 18.

The procedures for detecting the apoptosis by Hoechst staining are as follows:

1. IEC-6 cells were cultured in six-well plates at a density of 2×10⁵ cells/well.

2. After adhesion, the cells were treated with JAC4 (1 M) for 24 h.

3. The cells were subjected to X-ray radiation treatment.

4. The cells were stained with Hoechst 33258 (Beyotime, Shanghai) at 37° C. for 20 min.

5. The cells were washed thrice with PBS.

6. Fluorescence images were taken by confocal fluorescence microscopy. For each well, images were taken from 6 fields of view and recorded, and the mean values were calculated.

The procedures for determining apoptosis by ROS staining are as follows: The intracellular ROS content was determined using a ROS detection kit (Beyotime, Shanghai, China). IEC-6 cells were cultured in six-well plates at a density of 2×10⁵ cells/well. After adhesion, the cells were treated with JAC4 (1 μM) for 24 h, subjected to X-ray radiation, and cultured for another 24 h. The culture solution was completely discarded, and the cells were washed with PBS twice away from light. DCFH-DA was diluted with a serum-free culture medium. Not less than 600 μL of diluted DCFH-DA was added to each well, and the cells were incubated in a cell incubator for 25 min away from light. The cells were washed thrice with 1 mL/well of serum-free cell culture medium before the images were acquired by fluorescence microscopy.

As shown in FIG. 18, a large amount of ROS was produced and accumulated in IEC-6 cells after the X-ray radiation, the mitochondrial membrane potential was detected by JC-1 staining, and the red/green fluorescence ratio was calculated by using Image J. The value was 0.73±0.09 for the non-radiation control group, 0.32±0.10 for the vehicle+radiation group, and 0.49±0.05 for the JAC4+radiation group. After the X-ray radiation, the mitochondrial membrane potential in the cells was reduced, and the mitochondrial membrane potential after the JAC4 treatment was higher than that of the vehicle+radiation group. The membrane potential was reduced at the early stage of apoptosis, indicating that the early apoptosis was increased after the X-ray radiation but was relatively reduced after the JAC4 treatment. The apoptosis detection results by Hoechst staining show that the apoptosis rate was 0.02 t 0.01% for the non-radiation control group, 0.23±0.07% for the vehicle+radiation group, and 0.10 f 0.02% for the JAC4+radiation group. The results show that after the X-ray radiation, the apoptosis rate increased, while JAC4 treatment decreased the apoptosis rate as compared with the vehicle+radiation group.

The cell immunofluorescent images of γ-H2AX in different treatment groups were recorded after the IEC-6 cells were irradiated, and the mean fluorescence intensity of individual cells (***p<0.001) was measured using Image J. The results are shown in FIG. 19.

As can be seen from FIG. 19, the mean fluorescence intensity of the DNA double-strand break marker protein γ-H2AX was 17.17±1.47 for the non-radiation control group, 121.67±1.47 for the vehicle+radiation group, and 26.00±1.79 for the JAC4+radiation group. The results show that the γ-H2AX fluorescence increased after the X-ray radiation but decreased after JAC4 treatment.

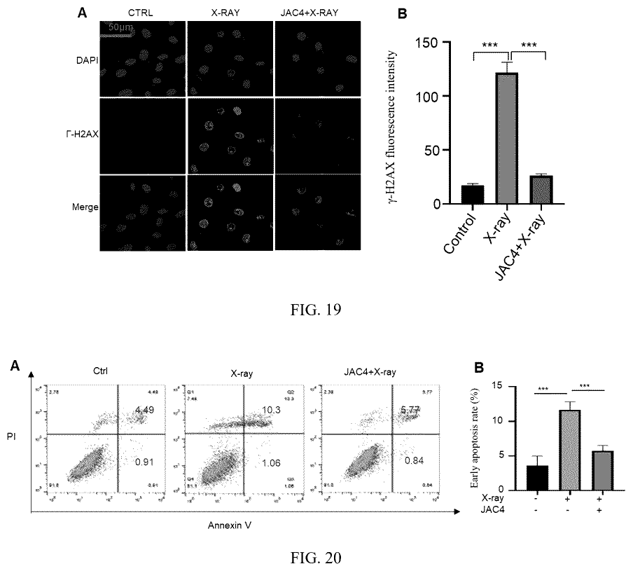

The apoptosis of IEC-6 cells was determined by Annexin V-PI staining and the early apoptosis rates of IEC-6 cells after different treatments were summarized (***p<0.001). The results are shown in FIG. 20. The procedures for the detection by Annexin V-PI staining are as follows: IEC-6 cells were seeded in a 6-well plate at 2×10⁵ cells/well. After adhesion, the cells were treated with 1 μM JAC4 or DMSO for 24 h and subjected to 12 Gy of X-ray radiation. After 24 h, the cells were harvested in PBS and analyzed using the Annexin V apoptosis detection kit (BD, FACS AriaIII, USA) on a flow cytometer.

As can be seen from FIG. 20, the early apoptosis rate was 3.57±1.41% for the non-radiation control group, 11.63±1.20% for the vehicle+radiation group, and 5.74±0.80% for the JAC4+radiation group. The results show that after the X-ray radiation, the early cell apoptosis rate increased, and JAC4 treatment decreased the early apoptosis rate.

### Example 14. Reduction of IEC-6 Cell Apoptosis by JAC4

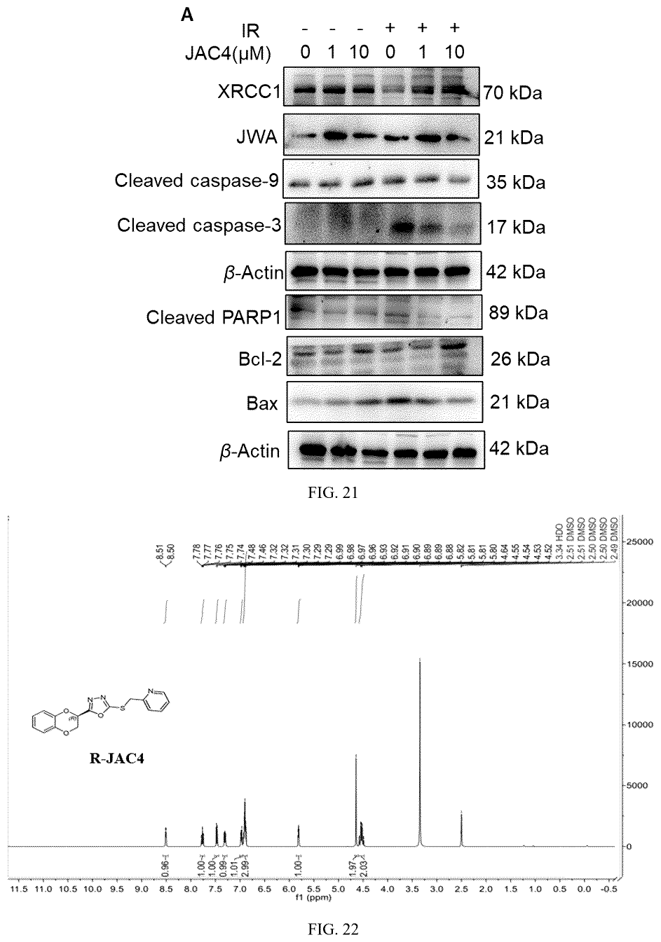

IEC-6 cells were pretreated with JAC4 (1 μM or 10 μM) or DMSO for 24 h and subjected to X-ray treatment (12 Gy). After 24 h, the cells were collected, and the total protein was extracted for Western blotting (with the same procedures as in Example 4) to determine the expression of JWA, spliced PARP1, Bcl-2, Bax, and spliced Caspase-3 in IEC-6 cells was examined. The results are shown in FIG. 21.

As can be seen in FIG. 21, the XRCC1 decreased in small intestine tissue and IEC-6 cells after the radiation, and the JAC4 treatment increased its expression level and promoted DNA repair. The ionizing radiation increased the levels of spliced pro-apoptotic protein PARP1, spliced Caspase-3, spliced Caspase-9 and Bax, and significantly reduced the levels of the anti-apoptotic proteins such as Bcl-2. Thus, the JAC4 treatment can resist radiation-induced apoptosis, as indicated by decreases in pro-apoptotic proteins as compared with the control group and increases in anti-apoptotic proteins as compared with the control group.

### Example 15. Preparation of R-JAC4

### Step 1: Preparation of Chiral Intermediate S1

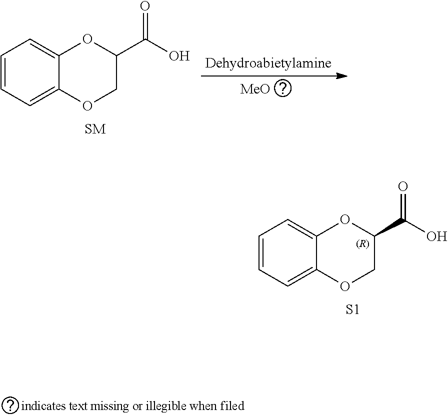

SM (10.2 g) was added to methanol (204 mL), and the mixture was stirred and heated with reflux. Dehydroabietylamine (9.68 g) was dissolved in methanol (102 mL). The solution of dehydroabietylamine in methanol was added dropwise at a high temperature, and the mixture was then stirred at the temperature for 30 min before the mixture was naturally cooled to 20-30° C. and a white solid was precipitated. The mixture was cooled in ice water to 0-10° C. for crystallization and filtered at reduced pressure. The filter cake was rinsed with methanol, dried, and dried in vacuum in an oven at 50° C. to give an off-white solid S1 (7.9 g; S:R=1.3:98.7).

The detection conditions of the S-to-R chiral molecule ratio are as follows:

Phase A: n-hexane; phase B: 0.1% diethylamine isopropanol; chromatographic column: Daicel CHIRALPAK AY-H (250 mm×4.6 mm, 5 μm); column temperature: 35° C.: flow rate: 1.0 mL/min; sample size: 5 μL; wavelength: 210 nm; concentration of the test sample: 1 mg/mL; solvent: ethanol; A:B (60:40, v/v), isocratic, 30 min.

S1 was then subjected to optical activity detection in the following conditions: sample pool temperature: 20° C.; sample concentration: 1.000 g/100 cm³; detection wavelength: 589 nm. The optical activity detection result was 59.098°.

### Step 2: Preparation of Intermediate S2

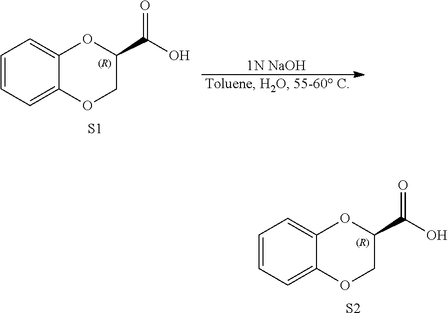

S1 was added to toluene (100 mL). The mixture was stirred for dispersion before 1 mol/L NaOH (40 mL) and purified water (50 mL) were added. The mixture was then stirred to give a white suspension, heated to 55-60° C., and stirred at the temperature for 30 min. The phases were separated before cooling. Toluene (50 mL) was added into the aqueous phase, and the mixture was stirred and let stand to separate the phases. The organic phase was discarded, and the aqueous phase was cooled in ice water. Concentrated hydrochloric acid (5 mL) was dropwise added to adjust the mixture to pH<1 before ethyl acetate (50 mL) was added. The mixture was stirred and the phases were separated. The aqueous phase was then extracted with ethyl acetate (50 mL) twice, and the organic phases were combined, dried over anhydrous sodium sulfate, concentrated to 5 mL, stirred, and cooled. 30 mL of n-heptane was added to precipitate a solid, and the mixture was cooled in ice water to 0-10° C. for crystallization and filtered at reduced pressure. The filter cake was rinsed with n-heptane and dried to give a pale yellow solid (2.63 g, two-step yield: 25.8%: S:R=1.07:98.9).

### Step 3: Preparation of Intermediate S3

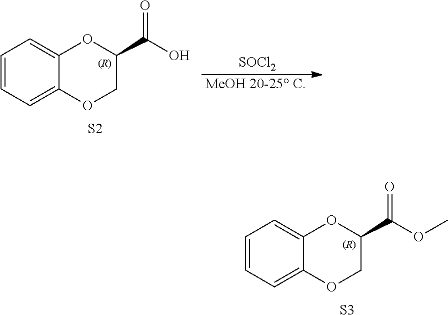

Methanol (12.5 mL) was added to a reaction flask before S2 (2.5 g) was added. The mixture was stirred until the solid was completely dissolved. SOCl₂ (1.82 g) was slowly and dropwise added while warming from 18° C., and the system was finally kept at 20-25° C. until the completion of the dropwise addition. The reaction system was incubated at the temperature for 2 h until the depletion of the starting materials. The mixture was concentrated at 30-35° C. before dichloromethane (12.5 mL) was added. Saturated aqueous NaHCO₃ solution (7.5 mL) was then added to adjust the aqueous phase to pH 8. The mixture was let stand to separate the phases. The organic phase was washed with 10,% aqueous NaCl solution (7.5 mL), and the phases were separated. The organic phase was dried over anhydrous sodium sulfate, concentrated before ethyl acetate was added, concentrated again at reduced pressure to 5 mL, and stirred and cooled until a solid was precipitated. n-Heptane (30 mL) was then added, and a large amount of solid was precipitated. The mixture was cooled in ice water to 0-10° C. for crystallization and filtered at reduced pressure. The filter cake was rinsed with i-heptane and dried to give a white solid (2.09 g, 77.6% yield; S:R=0.25:99.75).

### Step 4: Preparation of Intermediate S4

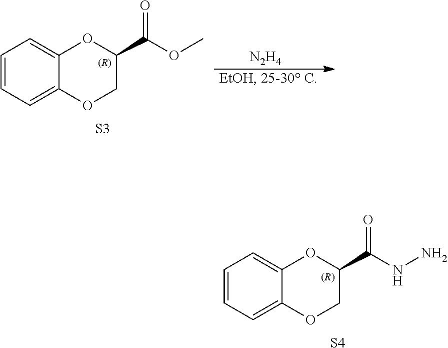

S3 (2.00 g), hydrazine hydrate (1.21 g), and ethanol (40 mL) were mixed, and the mixture was stirred and heated to 25-30° C. for reaction for 3 h. When no solid was precipitated, samples were taken for detection. When the starting materials were depleted, the mixture was concentrated by rotary evaporation to 6 mL at 35-40° C., stirred and cooled to 0-10° C. for crystallization for 1 h, and filtered at reduced pressure. The filter cake was rinsed with a small amount of absolute ethanol, and dried in vacuum to give a white solid (1.51 g, 75.5% yield; S:R=0.25:99.75).

### Step 5: Preparation of Intermediate S5

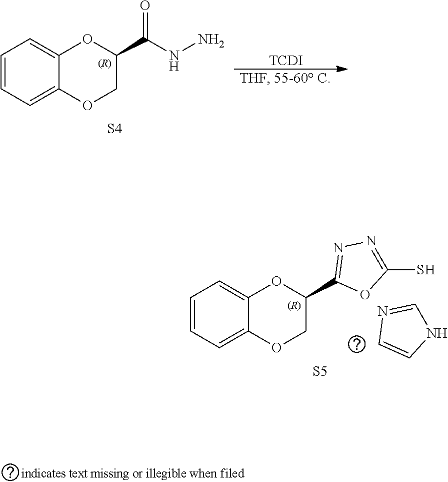

S4 (1.50 g), N,N-thiocarbonyldiimidazole (TCDI; 1.65 g), and tetrahydrofuran (15 mL) were added into a three-neck flask. The reaction system was stirred and heated to 55-60° C. for reaction for 5 h. When offline detection indicated the depletion of the starting materials, the reaction mixture was concentrated at 40-45° C. before ethyl acetate (7.5 mL) was added. The mixture was cooled to 0-10° C. for crystallization and filtered at reduced pressure. The filter cake was rinsed with ethyl acetate and dried in vacuum to give an off-white solid (1.16 g, 49.3% yield; S:R=0.43:99.56).

### Step 6: Preparation of R-JAC4

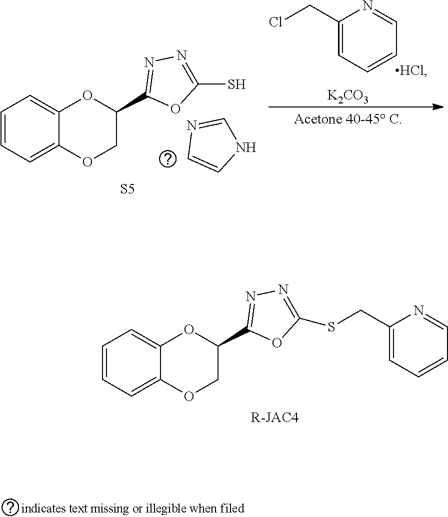

S5 (1.56 g), K₂CO₃ (2.13 g), acetone (15.6 mL), and 2-chloromethylpyridine hydrochloride (1.01 g) were added into a reaction flask. The reaction system was heated to 40-45° C. for reaction for 1.5-2 h. When offline detection indicated the depletion of the starting materials, the mixture was cooled to 20-25° C. before ethyl acetate (16 mL) and water (16 mL) were added. The mixture was stirred, and the phases were separated. The organic phase was washed with 15% NaCl solution, and the phases were separated. The organic phase was decolorized with 10% active carbon and filtered at reduced pressure. The filter cake was washed with ethyl acetate, and the filtrates were combined, concentrated to 5 mL, and stirred before n-heptane was dropwise added to precipitate a solid. The mixture was cooled to 0-10° C. for crystallization and filtered at reduced pressure. The filter cake was dispersed in acetone (5 mL) by stirring before purified water (15 mL) was added. The mixture was cooled to 0-10° C., triturated, and filtered at reduced pressure. The filter cake was dried in vacuum to give an off-white solid powder (1.34 g, 79.5% yield).

DMSO-D6δ_{H}: 8.51 (d, 0.1=4.3 Hz, 1H), 7.76 (d, J=7.7, 1.8 Hz, 1H), 7.47 (d, 1=7.8 Hz, 1H), 7.36-7.26 (m, 1H), 7.01-6.95 (m, 1H), 6.94-6.86 (m, 3H), 5.81 (dd, J=4.9, 2.9 Hz, 1H), 4.64 (s, 2H), 4.59-4.48 (m, 2H) (FIG. 22);

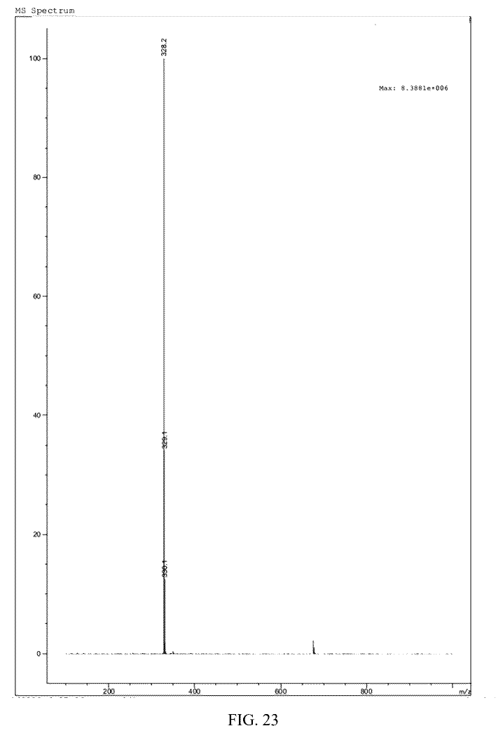

LC/MS(m/z, MH⁺): 328.2 (FIG. 23).

### Example 16. Preparation of S-JAC4

The structure of S-JAC4 is shown in the following formula (II):

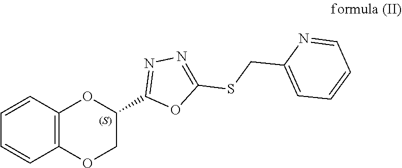

S-JAC4 was obtained by chiral column resolution from a JAC4 racemate, and the conditions for resolution are as follows:

Instrument: MGII preparative SFC (SFC-1)

Column: ChiralPak AS, 250×30 mm, I.D. 10 μm

Mobile phase: A was CO₂, and B was isopropanol (0.1% NH₃H₂O)

Gradient: B 35%

Flow rate: 80 mL/min

Back pressure: 100 bar

Column temperature: 38° C.

Wavelength: 220 nm

Cycle time: 5 min

Sample preparation: The compound was dissolved in 100 mL of methanol/DCM

Injection: 2 mL/injection.

Post-treatment: The separated fractions were dried on a rotary evaporator at a bath temperature of 40° C. to give the desired isomer.

The synthesis route of the JAC4 racemate is as follows:

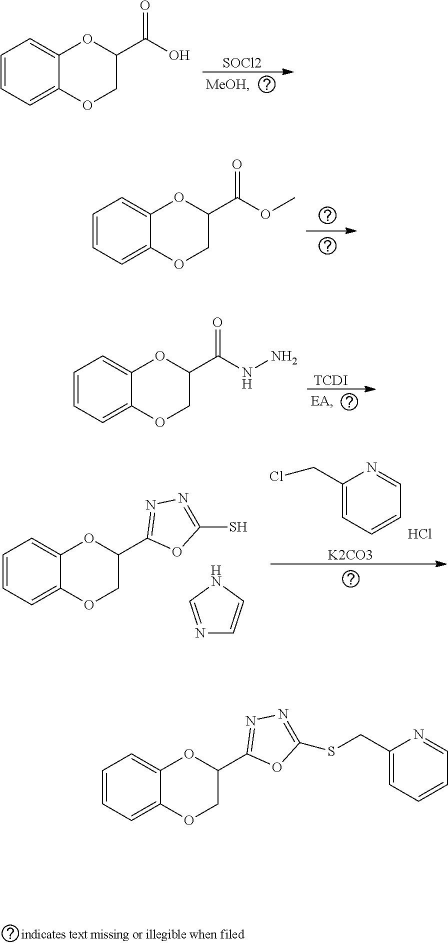

### Example 17. Pharmacokinetics of R-JAC4 and S-JAC4

### Animals:

Healthy adult female BALB/c mice, purchased from Beijing Vital River Laboratory Animal Technology Co., Ltd., animal production license number. SCXK (Beijing) 2016-0006. The mice were randomized into 2 groups each containing 3 mice and receiving intragastric administration and intravenous administration, respectively.

### Formulation:

Proper amounts of compounds R-JAC4 and S-JAC4 were dissolved in a mixture of, in portions by volume, 5% of DMSO, 40% triglyceride (TG), and 55% of an aqueous solution containing 7.5% (w/v) sulfobutyl-β-cyclodextrin sodium salt, so as to give a 0.5 mg/mL solution for intravenous administration; Similarly, a 1 mg/mL solution was prepared for intragastric administration.

### Administration:

Intravenous group: BALB/c mice were deprived of food overnight and the treatment was administered intragastrically at a dose of 3 mg/6 mL/kg.

Intragastric group: BALB/c mice were deprived of food overnight and the treatment was administered through the tail vein at a dose of 10 mg/10 mL/kg.

### Procedures:

After the mice received the intragastric or intravenous dose, 40 μL of orbital blood was collected into an EDTA-2K anticoagulation tube at 5 min, 15 min, 30 min, 1 h, 2 h, 6 h, and 24 h post-dose, and plasma was separated by centrifugation at 12000 rpm for 5 min at 4° C. The separated plasma was preserved at −20° C.

The plasma concentration of the compound in mice receiving different intravenous or intragastric doses was measured by the following specific procedures: The plasma samples were thawed at room temperature, and vortexed for 1 min. 10 μL of the sample was transferred to a 2-mL 96-well plate, and 50 μL of internal standard solution (tolbutamide in acetonitrile) and 50 μL of precipitator (acetonitrile:methanol=7:3 (v/v)) were added. The plate was shaken at 1000 rpm for 3 min and centrifuged at 4000 rpm for 15 min. 20 μL of the supernatant was transferred to a 2 mL 96-well plate, before 280 μL of water was added for dilution. The plate was shaken at 1000 rpm for 3 min and the samples were injected for analysis. The mice were euthanized after modeling.

LC-MS/MS conditions: chromatographic column: Waters T3 1.8 μm (2.1 mm×30 mm), mobile phase A: 0.1% aqueous formic acid; mobile phase B: 0.1% formic acid in acetonitrile; column temperature: 40° C.; sample injection amount: 5 μL; flow rate: 0.6 mL/min. The gradient elution procedures are shown in Table 1.

TABLE 1
Gradient elution procedures

| Time (min) | Mobile phase A (%) | Mobile phase B (%) |
|---|---|---|
| 0 | 80 | 20 |
| 0.1 | 80 | 20 |
| 0.7 | 2 | 98 |
| 1.0 | 2 | 98 |
| 1.01 | 80 | 20 |
| 1.5 | 80 | 20 |

The pharmacokinetic results of the test compounds are detailed in Tables 2 and 3.

TABLE 2
Pharmacokinetics after a single intravenous dose
of compound (Mean ± SD, n = 3)

| | C0 | C1 (mL/ | T_{1/2} | AUClast | Vss |
|---|---|---|---|---|---|
| Compound | (ng/mL) | min/kg) | (hr) | (hr*ng/mL) | (L/Kg) |
| R-JAC4 | 1679 ± | 140 ± | 0.600 ± | 374 ± | 3.64 ± |
| | 513.1 | 47.4 | 0.242 | 116.1 | 0.251 |
| S-JAC4 | 1651 ± | 121 ± | 1.93 ± | 410 ± | 7.72 ± |
| | 314.8 | 13.69 | 1.048 | 47.39 | 4.82 |

TABLE 3
Pharmacokinetics after a single intragastric dose
of compound (Mean ± SD, n = 3)

| | Cmax | Tₘₐₓ | | AUClast | |
|---|---|---|---|---|---|
| Compound | (ng/mL) | (hr) | T_{1/2} (hr) | (hr*ng/mL) | F (%) |
| R-JAC4 | 176 ± | 0.083 ± | 0.378 ± | 49.5 ± | 3.94 ± |
| | 14.57 | 0 | 0.026 | 5.667 | 0.448 |
| S-JAC4 | 48.7 ± | 0.083 ± | 0.451 ± | 17.2 ± | 1.32 ± |
| | 13.46 | 0 | 0.117 | 3.514 | 0.206 |

According to the bioavailability equation F=(AUC-po/Dose-po)/(AUC-iv/Dose-iv), where AUC-po denotes the area under the oral dose curve, AUC-iv denotes the area under the intravenous injection curve, Dose-po denotes the oral dose, and Dose-iv denotes the intravenous dose. The calculation shows that the oral bioavailability of R-JAC4 is higher than that of S-JAC4.

### Example 18. Pharmacological and Pharmacodynamic Effects of R-JAC4 on Radiation Enteritis Mouse Model

Male C57/BL6 mice aged 10 weeks were selected and randomized into 4 groups before the start of the experiment, including a normal control group (i.e., mice that did not receive any treatment and radiation), a vehicle control group, a R-JAC4 10 mg/kg treatment group, a R-JAC4 3 mg/kg treatment group. The formulation of the vehicle for R-JAC4 was polyethylene glycol:ethanol:physiological saline=40:7.5:52.5, v/v/v. The vehicle group and the treatment groups were given the vehicle or treatment at the same volume by the same route.

The vehicle was administered to the vehicle control group intragastrically, and R-JAC4 10 mg/kg and R-JAC4 3 mg/kg interventions were given to the R-JAC4 treatment groups intragastrically. The treatment started 7 days before the X-ray radiation and was administrated once daily until the day before the end of study, for a total of 7 doses.

X-ray radiation: The mice were anesthetized intraperitoneally and exposed to ABI using an X-ray irradiator (12 Gy, 1.25 Gy/min). The irradiated region was a 3 cm region above the iliac joint below the chest, so as to induce gastrointestinal radiation syndrome.

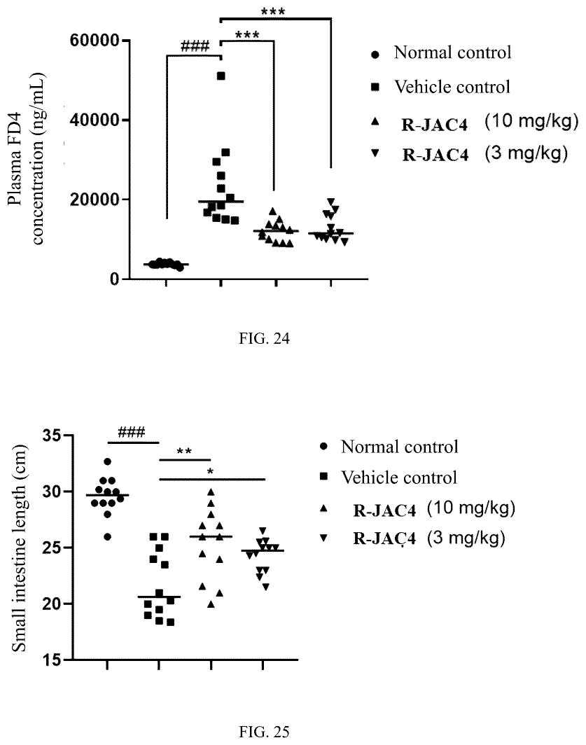

Permeability detection in the small intestine: On day 4 post-radiation, FITC-dextran 4 kDa, abbreviated as FD4, was given to the mice intragastrically at a dose of 0.6 mg/g body weight. Peripheral blood was collected in an anticoagulation tube after 4 h, and the plasma was collected by centrifugation. The samples were added to a 96-well plate at 100 μL, and the fluorescence intensity was measured (excitation wavelength: 485 nm, emission wavelength: 535 nm). The FD4 concentrations of the samples were calculated by regression according to a standard curve. The results are shown in FIG. 24.

The results show that the permeability of fluorescent molecule FD4 in the normal vehicle control group was higher than those in the treatment groups, and the compound R-JAC4 at 10 mg/kg and 3 mg/kg showed a dose-dependent down-regulation effect on the concentration of fluorescein FD4 in the plasma in the radiation enteritis model mice, indicating that the administration of R-JAC4 can reduce the damage to the mouse intestinal epithelium and preserve the barrier function of the intestinal epithelium.

Small intestine length measurement: At the end of study, the mice were euthanized and dissected. The whole small intestine was taken and placed on a piece of coordinate paper to measure the length of the small intestine. The results are shown in FIG. 25.

The results show that compound R-JAC4 is effective in reducing the shortening extent of the small intestine in a dose-dependent manner.

### Example 19. Study on R-JAC4 in Terms of Anti-Inflammatory and Anti-Oxidant Properties

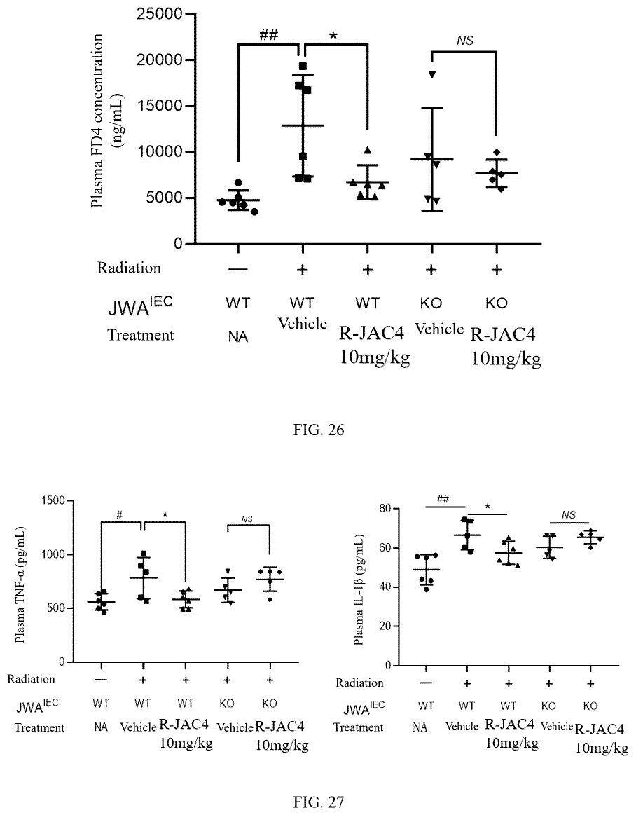

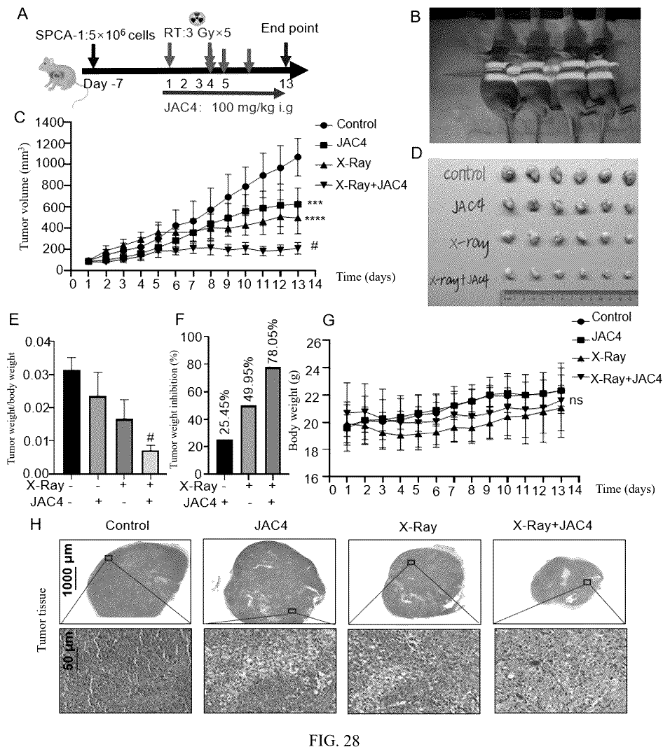

Intestinal epithelial cell JWA-knockout mice (JWA^{IEC KO}) and their wild type littermates (JWA^{IEC WT}) were subjected to radiation enteritis modeling in the same manner as in Example 17 and were given R-JAC4 10 mg/kg treatment. In addition to the detection of FD4 concentration, the plasma was collected from the mice at the end of study to detect the plasma proinflammatory cytokine level by ELISA and the activity of antioxidant-related enzymes by biochemical tests. The results are shown in FIGS. 26 and 27.

The results show that: the plasma FD4 level was significantly downregulated by R-JAC4 10 mg/kg treatment in the JWA^{IEC WT} mouse radiation enteritis model, while no significant change in FD4 levels was observed in JWA^{IEC KO} mice as compared with the corresponding vehicle control group; R-JAC4 10 mg/kg treatment in the JWA^{IEC WT} mouse radiation enteritis model significantly reduced the plasma levels of proinflammatory cytokines TNF-α and IL-1β, while the R-JAC4 treatment exhibited no significant changes in JWA^{IEC KO} mice as compared with the corresponding vehicle control group; the R-JAC4 10 mg/kg treatment significantly increased the activity of plasma glutathione peroxidase and catalase in the JWA^{IEC WT} mouse radiation enteritis model, while the R-JAC4 treatment exhibited no significant changes in JWA^{IEC KO} mice as compared with the corresponding vehicle control group. Therefore, R-JAC4 exerts anti-inflammatory and antioxidant functionality through JWA-related signaling pathways, thus possessing therapeutic effects in mice with radiation enteritis.

In summary, in the present disclosure, in one aspect, JAC4 can increase the transcription of XRCC1 and inhibit the degradation of XRCC1 by activating JWA, thereby repairing X-ray-induced DNA damage in small intestine cells via the BER signaling pathway. In another aspect, JWA, as an effective response gene, improves the expression of glutathione peroxidase and superoxide dismutase, reduces the generation of malondialdehyde in cells, and thus reduces the reactive oxygen species content in cells. In addition, JWA also protects the mitochondrial membrane, reduces the release of cytochrome C, inhibits the pro-apoptotic protein Bax, activates the anti-apoptotic protein Bcl-2, and finally suppresses the generation amount of spliced Caspase-9 and Caspase-3, thus effectively reducing the occurrence of mitochondrial apoptosis in small intestine cells and protecting the irradiated small intestine epithelial cells.

### Example 20. Synergistic Effect of JAC4 on X-Ray Radiation Therapy in a Subcutaneous Lung Cancer Xenograft Model

SPCA-1 cells in the logarithmic growth phase were subcutaneously injected into male BALB/C nude mice aged 4-5 weeks at 5×10⁶ cells/100 μL flush with the lung. When the tumor volume reached 60-100 mm³ (tumor volume=length×width²/2), the mice were randomized into a vehicle control group, a 100 mg/kg JAC4 group (daily treatment), a 3 Gy×5 X-ray radiation group (5 consecutive days of radiation), and a 100 mg/kg JAC4+3 Gy×5 X-ray radiation group, each containing 6 mice. The formulation of the vehicle for JAC4 was polyethylene glycol:ethanol:physiological saline=40:7.5:52.5, v/v/v. The vehicle group and the treatment groups were given the vehicle or the treatment at the same volume by the same route, respectively. The mice were kept anesthetized by isoflurane through a mask in a supine posture. The irradiation range was adjusted to the tumor and the whole lung with a width of about 2 cm through the width indicating lamp and the beam limiting device of the system. The chamber was closed, and the irradiation time was set to about 126 s according to the dose rate to enable the total dose to reach 3 Gy. The irradiator was turned on for the irradiation. 100 mg/kg JAC4 was administered intragastrically daily. The body weight and tumor volume were measured daily and the modeling was terminated on day 13 post-dose.

After the modeling was finished, the tumor tissue was subjected to HE staining according to the following specific procedures: 3 mice were randomly selected to provide the tumor tissue for HE staining in each group, and 6 fields of view were randomly selected for inspection.

As can be seen from FIG. 28, the results in the SPCA-1 cell xenograft mouse model show that: the tumor volumes of the 100 mg/kg JAC4 monotherapy and the radiation monotherapy were significantly smaller than the control group, while the tumor inhibitory effect of the combination therapy group was superior to those of all other groups (FIGS. 28C and 28D). The tumor-to-body weight ratio was significantly reduced in mice in the combination therapy group as compared with the X-ray monotherapy group (FIG. 28E). The tumor inhibition rates for the 100 mg/kg JAC4 therapy, radiation therapy, and combination therapy were 25.45%, 49.95%, and 78.05%, respectively, as compared with the control group (FIG. 28F). In addition, no significant changes were observed in body weight among all groups, and no death occurred, demonstrating that JAC4 has no significant adverse effects on mice (FIG. 28G). In addition, the H&E staining results in tumor tissues of the xenograft animals show that JAC4 in combination with X-ray therapy exhibited a sparse cell arrangement and numerous gaps in tumor tissues as compared with other treatment groups, demonstrating that JAC4 in combination with X-ray promotes the necrosis of tumor tissues and results in significant tissue necrosis in the central region of the tumor mass (FIG. 28H). The above results show that JAC4 exerts a synergistic effect on the X-ray therapy in subcutaneous SPCA-1 xenograft tumor-bearing mouse model.

### Example 21. Inhibitory Effects of JAC4 on X-Ray-Induced Inflammatory Response and Pulmonary DNA Damage in Lung Cancer Xenograft Mice

After the modeling in Example 20 was completed, the inflammation level and DNA damage in the lung tissue were detected by the following specific procedures: 3 mice were randomly selected from each group to provide the lung tissue. About 20-30 mg of lung tissue was collected from each mouse in a centrifuge tube. The total RNA was isolated and extracted using the TRIzol reagent (Invitrogen) for RT-PCR (Reverse Transcription-Polymerase Chain Reaction) detection. The RNA was reversely transcribed into cDNA using a reverse transcription kit from Vazyme. Reaction system: 42° C., 2 min; 37° C., 15 min; 85° C., 5 s. The samples were preserved in a refrigerator at −2° C. or −80° C. for later use. 30-35 PCR amplification cycles were performed. The data were analyzed by the ΔΔCt method using glyceraldehyde-3-phosphate dehydrogenase (GAPDH) as an internal standard and the AceQ qPCR SYBR Green Master Mix fluorescent quantitative PCR reagent from Vazyme on the 7900HT high throughput flash real-time fluorescent quantitative PCR system from Thermo Fisher Scientific for PCR assay. The results are shown in FIG. 29, with the following specific conditions:

### The Primer Sequences:

GAPDH:
Forward:
5′-CATCACTGCCACCCAGAAGACTG-3′;
Reverse:
5′-ATGCCAGTGAGCTTCCCGTTCAG-3′;
IL-10:
Forward:
5′-CGGGAAGACAATAACTGCACCC-3′;
Reverse:
5′-CGGTTAGCAGTATGTTGTCCAGC-3′;
TNF-α:
Forward:
5′-GGTGCCTATGTCTCAGCCTCTT-3′;
Reverse:
5′-GCCATAGAACTGATGAGAGGGAG-3′;
IL-1β:
Forward:
5′-TGGACCTTCCAGGATGAGGACA-3′;
Reverse:
5′-GTTCATCTCGGAGCCTGTAGTG-3′;
TGF-β1:
Forward:
5′-TGATACGCCTGAGTGGCTGTCT-3′;
Reverse:
5′-CACAAGAGCAGTGAGCGCTGAA-3′.

### Amplification System:

| Reagent | Amount | |
|---|---|---|
| Green Master Mix | 5 | μL |
| Primer1 (10 μM) | 0.2 | μL |
| Primer2 (10 μM) | 0.2 | μL |
| 50 × ROX Reference Dye 1 | 0.2 | μL |
| cDNA | 1 | μL |
| ddH₂O | 3.4 | μL |

### Amplification Conditions:

| Step | Cycles | Temperature | | Reaction time | |
|---|---|---|---|---|---|
| Stage 1 | 1 | 95° | C. | 5 | min |
| Stage 2 | 40 | 95° | C. | 10 | s |
| | | 6° | C. | 30 | s |
| | | 95° | C. | 15 | s |
| Stage 3 | 1 | 60° | C. | 60 | s |
| | | 95° | C. | 15 | s |

As can be seen in FIG. 29, the RT-PCR results showed that the mRNA level of anti-inflammatory factor IL-10 in the X-ray group was 0.38 folds that in the control group (P<0.05), while the level in the JAC4+X-ray group was 2.91 folds that of the X-ray group (P<0.05) (FIG. 29A); the mRNA level of proinflammatory factor TNF-α in the X-ray group was 1.33 folds that in the control group (P<0.05), while the level in the JAC4+X-ray group was 0.63 folds that of the X-ray group (P<0.01) (FIG. 29B); the mRNA level of proinflammatory factor TGF-β1 in the X-ray group was 1.38 folds that in the control group (P<0.05), while the level in the JAC4+X-ray group was 0.71 folds that of the X-ray group (P<0.01) (FIG. 29C); the mRNA level of proinflammatory factor IL-1P in the X-ray group was 1.90 folds that in the control group (P<0.001), while the level in the JAC4+X-ray group was 0.43 folds that of the X-ray group (P<0.001) (FIG. 29D); in addition, the HE staining results of lung tissues in all groups show thickened lung interstitium, reduced alveolar septal space, and a large amount of infiltrating inflammatory cells in the X-ray treatment group, while the alveolar wall was thinned after the JAC4 treatment in combination with the X-ray therapy, suggesting reduced inflammatory cell infiltration (FIG. 29E). The above results indicate that JAC4 reduces the X-ray-induced inflammatory response.

In order to verify the protection effect of JAC4 against X-ray-induced DNA damage in lung tissues, 3 mice were randomly selected from each group to provide the lung tissue for immunofluorescence staining, and proteins were extracted from the lung tissues of the other 3 mice for western immunoblot to detect the expression levels of JWA, γ-H2AX, and p-p65, thereby verifying the relation between the targets of JAC4 and the reduction of DNA damage and apoptotic damages in the lung tissues. The western blot procedures are detailed in Example 4, and the immunofluorescence staining procedures are as follows:

1. Dewaxing and hydration: Prepared paraffin sections were dewaxed in an oven at 60° C. for 2-3 h to ensure a tight attachment between the section and the slide; the sections were then dewaxed by soaking in xylene I, 30 min→xylene II, 30 min→xylene III, 30 min→absolute ethyl alcohol I, shaker, 5 min→absolute ethyl alcohol II, shaker, 5 min→95% ethyl alcohol, shaker, 5 min→85% ethyl alcohol, shaker, 5 min→75% ethyl alcohol, shaker, 5 min→PBS, shaker, 5 min×3.

2. Antigen retrieval: The washed sections were boiled in 500 mL of 1× antigen retrieval solution (20× sodium citrate buffer 25 mL, pH 6.0, diluted to 1× in 475 mL of PBS), then taken out 10 min after the solution started boiling, and cooled to room temperature.

3. Cleaning and blocking: The sections were washed with PBS for 5 min thrice on a shaker. Droplets on the sections were removed carefully without overdrying. The edges of the sections were marked with an immunofluorescent pen, and a blocking buffer (centrifuged at 12,000 g for 5 min) was dropwise added to completely cover the sections. The sections were blocked for 1 h at room temperature.

4. Cleaning and co-incubation with primary antibody: After removing the blocking buffer, the sections were washed with PBS for 5 min thrice on a shaker; the primary antibody (centrifuged at 6,000 g for 5 min) was added dropwise to cover the sections, and the sections were incubated into a humidified chamber overnight at 4° C.

5. Cleaning and co-incubation with secondary antibody: The sections were washed with PBS for 5 min thrice on a shaker before the fluorescent secondary antibody (centrifuged at 6,000 g for 5 min) was added dropwise away from light. The sections were incubated at room temperature for 1 h.

6. Cleaning, nucleus staining, and photographing: The whole procedure was performed away from light. The sections were washed with PBS for 5 min thrice on a shaker. An anti-fluorescence quenching blocker containing DAPI was added dropwise. The cover glass was mounted for sealing and slowly from one side to avoid bubbles. The sealed slices were incubated in a humidified chamber for 15 min and continuously photographed to give images under a laser confocal microscope.

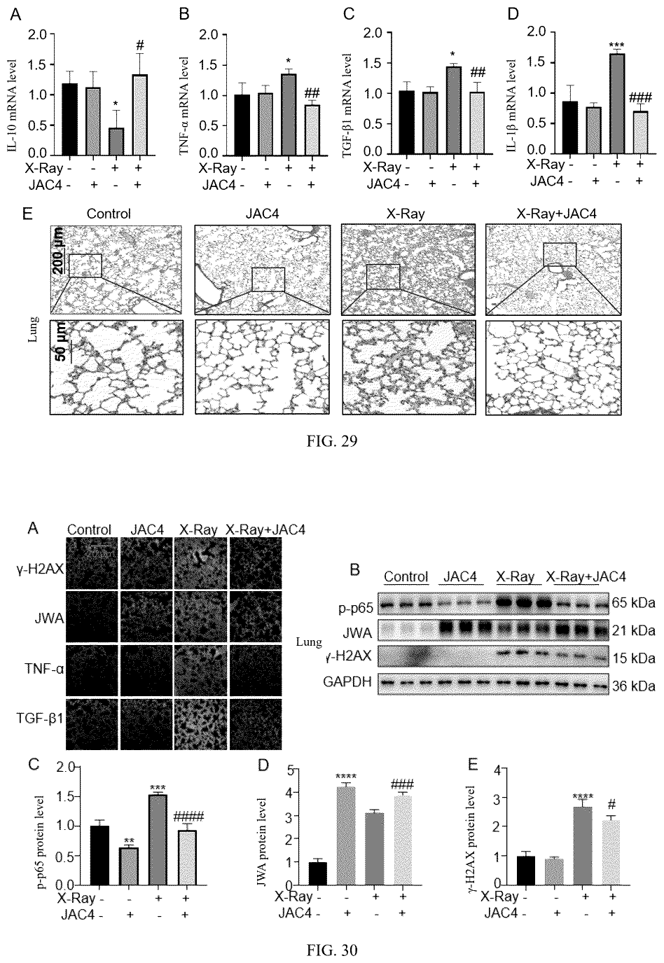

As can be seen from the results of immunofluorescence study in FIG. 30A, JAC4, X-ray, and JAC4+X-ray groups had higher JWA fluorescence intensity as compared with the control group; the fluorescence intensities of γ-H2AX and inflammatory factors TNF-α and TGF-β1 in the X-ray group were significantly increased; the fluorescence intensities of γ-H2AX, TNF-α, and TGF-β1 in the JAC4+X-ray group were significantly decreased as compared with the X-ray group. Proteins were extracted from lung tissues in all groups. The immunoblot assay results, as shown in FIGS. 30B-30E, demonstrate that the p-p65 protein levels in the JAC4 group and the X-ray group were 0.63 folds (P<0.01) and 1.53 folds (P<0.001) that in the control group, respectively, while the level in the JAC4+X-ray group was 0.61 folds (P<0.0001) that in the X-ray group; the JWA protein levels in the JAC4 group and the X-ray group were 4.23 folds (P<0.0001) and 3.11 folds (P<0.0001) that in the control group, respectively, while the level in the JAC4+X-ray group was 1.25 folds (P<0.01) that in the X-ray group; the γ-H2AX protein levels in the JAC4 group and the X-ray group were 0.91 folds (P=0.90) and 2.68 folds (P<0.0001) that in the control group, respectively, while the level in the JAC4+X-ray group was 0.83 folds (P<0.05) that in the X-ray group. The results are the same as those of the immunofluorescence study. The above results show that JAC4, in combination with X-ray, promotes the expression of JWA, inhibits p-p65, and further reduces the X-ray-induced DNA damage in lung tissues.

### Example 22. Effect of JAC4 in Combination with X-Ray Radiation on Human Lung Adenocarcinoma Cells SPCA-1 and Human Normal Lung Epithelial Cells BEAS-2B

SPCA-1 and BEAS-2B cells were cultured in a 90%/DMEM+10% FBS+100 μg/mL ciprofloxacin culture medium. The SPCA-1 and BEAS-2B cells were pretreated for 24 h with 10 μM JAC4 and subjected to 4 Gy X-ray treatment for 24 h and then CCK-8 detection, so as to explore the influence of JAC4 in combination with X-ray on the viability of SPCA-1 and BEAS-2B cells. The specific procedures are as follows:

1. SPCA-1 and BEAS-2B cells in the exponential growth phase were plated in a 96-well plate at 5,000 cells/well in triplicate for each group.

2. After 24 h of culture, when the cells fully adhered to the container wall and returned to normal morphology, the cells were treated with X-ray radiation of 0, 4, 8, 12, 16, or 20 Gy or JAC4 at 0, 1, 5, 10, 20, or 50 μM.

3. After 24 h of treatment, CCK-8 reagent was added at 10 μL/well, and the cells were incubated in a cell incubator for 1 h. The absorbance (OD) at 450 nm was measured.

4. Cell viability equation: cell viability (%)=(OD_{control}−ODₜᵣₑₐₜₘₑₙₜ)×100%/(OD_{control}−OD_{blank}).

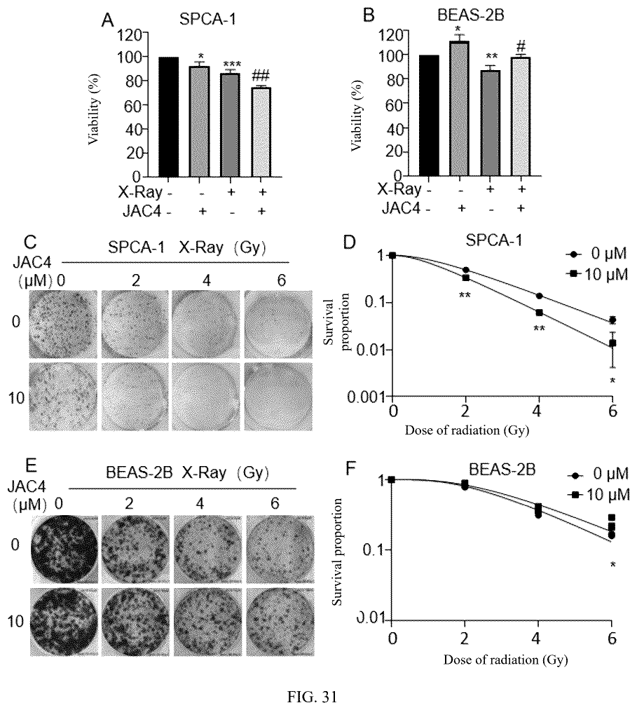

As shown in FIG. 31, the viability of SPCA-1 and BEAS-2B cells was reduced after X-ray treatment; the combination of JAC4 with X-ray significantly inhibited the viability of SPCA-1 cells (FIG. 31A), while the combination of JAC4 with X-ray alleviated the inhibitory effect of X-ray on BEAS-2B cell viability (FIG. 31B).

The cell proliferation capacity was determined by a plate colony formation experiment, with the following specific procedures.

1. SPCA-1 and BEAS-2B cells (or SPCA-1 and BEAS-2B cells transfected with JWA-knockdown plasmids) in the exponential growth phase were plated in 6-well plates at a specific amount. The number of plated cells is as follows

| | Dose of | Number of |
|---|---|---|
| Cell species | radiotherapy (Gy) | seeded cells |
| SPCA-1 | 0 | 500 |
| BEAS-2B | 2 | 500 |
| | 4 | 1000 |
| | 6 | 1500 |

2. After the cells adhered to the container wall, the cells were treated (or not treated) with 10 M JAC4 and treated with X-ray of 0, 2, 4, or 6 Gy. The medium was replaced with a fresh medium containing JAC4 at the corresponding concentration once every 2 days (or the cells were treated with X-ray of 0, 2, 4, or 6 Gy after adhesion, and the medium was replaced with a fresh medium once every 2 days).

3. After the cells were cultured for 10-14 days, colonies were visually observed. The culture medium was discarded, and the cells were fixed with methanol for 10-20 min, stained with crystal violet for 10 min, slowly washed with flowing clear water thrice, photographed, and counted for colonies containing>50 cells.

4. The colony forming efficiency (CFE)=(number of colonies formed/number of seeded cells) 100% was calculated, and the cell survival fraction (SF)=(colony forming efficiency of treatment group/colony forming efficiency of 0 Gy group)×100% was calculated from the colony forming efficiency.

The results show that JAC4 in combination with X-ray therapy decreased the number of colonies formed by SPCA-1 cells (FIG. 31C), but increased the number of colonies formed by BEAS-2B cells (FIG. 31E). A radiosensitization curve was fitted using the one-click multi-target model, which indicates that 10 μM JAC4 in combination with X-ray significantly reduced the survival of SPCA-1 cells as compared with the X-ray monotherapy group (FIG. 31D), and 10 μM JAC4 in combination with X-ray slightly increased the survival of BEAS-2B cells as compared with the X-ray monotherapy group (FIG. 31F). The results show that JAC4 in combination with X-ray inhibited the viability and proliferation of SPCA-1 cells, and increased the viability and proliferation of BEAS-2B cells.

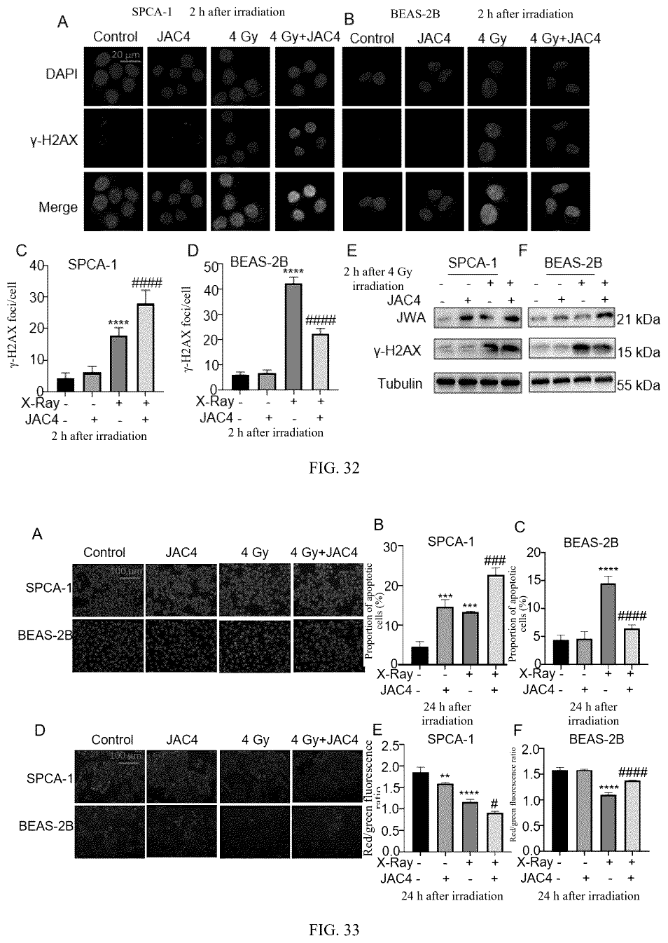

To investigate the effect of JAC4 in combination with X-ray on DSBs in SPCA-1 and BEAS-2B cells, SPCA-1 and BEAS-2B cells were pretreated with 10 μM JAC4 for 24 h and 4 Gy X-ray for 2 h, and then subjected to γ-H2AX fluorescent labeling and Western immunoblot (see Example 4 for detailed procedures). The results, as shown in FIG. 32, show that a large number of γ-H2AX foci were found in the nucleus after 4 Gy X-ray radiation, both in SPCA-1 cells and BEAS-2B cells, as compared with the control group. JAC4 in combination with X-ray treatment resulted in an increase in γ-H2AX foci in SPCA-1 cells as compared with the X-ray monotherapy group (FIGS. 32A and 32C). JAC4 in combination with X-ray significantly reduced the formation of γ-H2AX foci in BEAS-2B cells as compared with the X-ray treatment group (FIGS. 32B and 32D). The results of Western blot show that as compared with the control group, the 4 Gy X-ray radiation increased the γ-H2AX expression level. JAC4 in combination with X-ray treatment increased the JWA expression level and the γ-H2AX expression level in SPCA-1 cells as compared with the X-ray monotherapy group (FIG. 32E); in BEAS-2B cells, JAC4 in combination with X-ray increased the JWA expression level and decreased the γ-H2AX protein expression level (FIG. 32F). The above results indicate that JAC4 in combination with X-ray increases the DSBs of X-ray in SPCA-1 and alleviates the DSBs in BEAS-2B cells by activating the expression of JWA.

Severe DSBs may cause apoptosis. SPCA-1 and BEAS-2B cells were plated and cultured for 24 h before they were treated with 10 μM JAC4 for 24 h and 4 Gy X-ray for 24 h. The cells were stained with Hoechst 33342 (see Hoechst staining for apoptosis detection in Example 13) to evaluate the effect of JAC4 in combination with radiation on apoptosis. The results, as shown in FIG. 33, show that X-ray increased the apoptosis of SPCA-1 cells as compared with the control group, and JAC4 increased the pro-apoptotic effect of X-ray on SPCA-1 cells (FIGS. 33A and 33B). In contrast, in BEAS-2B cells, X-ray increased the apoptosis of BEAS-2B cells as compared with the control group, while JAC4 decreased the pro-apoptotic effect of X-ray on BEAS-2 cells (FIGS. 33A and 33C). The decrease in mitochondrial membrane potential (JC-1) is a marker event in the early stage of apoptosis. The mitochondrial membrane potential was measured using fluorescent probe JC-1. The red fluorescence indicates a high mitochondrial membrane potential leading to JC-1 aggregate formation, and the green fluorescence indicates the mitochondrial membrane potential depolarization. The fluorescence ratio of red to green reflects the mitochondrial membrane potential and the number of early apoptotic cells. The mitochondrial membrane potential assay results show that X-ray caused a decrease in mitochondrial membrane potential, indicating an increase in the number of early apoptotic cells, and that JAC4 in combination with X-ray treatment aggravated the decrease in mitochondrial membrane potential in SPCA-1 cells (FIGS. 33D and 33E). In contrast, JAC4 reversed the X-ray-induced decrease in mitochondrial membrane potential in BEAS-2B cells (FIGS. 33D and 33F).

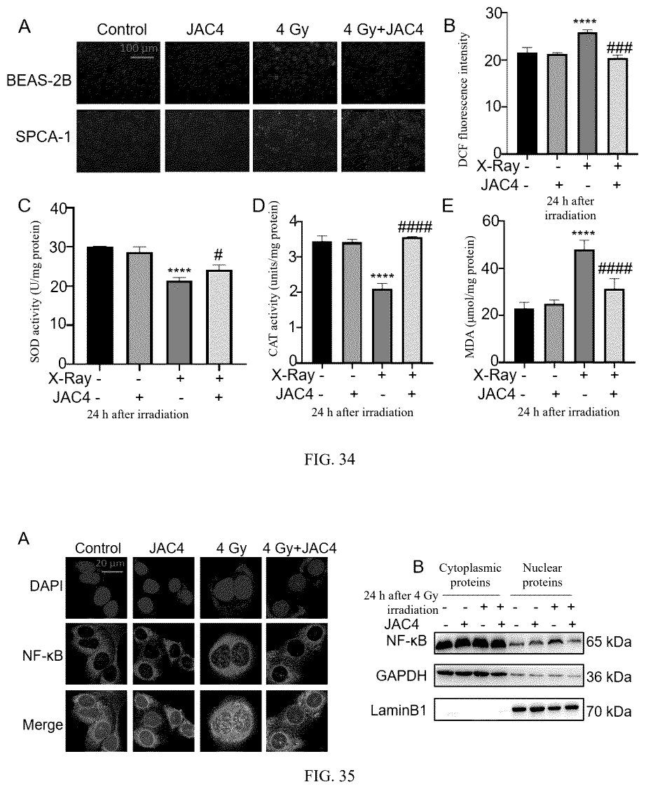

The large amount of reactive oxygen species (ROS) induced by X-ray and the sustained oxidative stress reaction may cause DNA damage. Superoxide dismutase (SOD) and catalase (CAT) exert antioxidant effects, and the lipid oxidation product malondialdehyde (MDA) and ROS can reflect the oxidative stress level in vivo. The intracellular ROS level was detected using the fluorescent probe DCFH-DA. As can be seen from the ROS assay results in FIG. 34, X-ray increased the intracellular ROS level, while JAC4 in combination with X-ray significantly reduced the ROS level in BEAS-2B cells. JAC4 had no significant effect on the ROS level in SPCA-1 cells. The supernatant of BEAS-2B cells was extracted and subjected to an MDA assay and an activity assay for CAT and SOD. The results show that in BEAS-2B cells of the DMSO, JAC4, X-ray, and JAC4+X-ray groups, the SOD activity was 30.00±0.14, 28.67±1.36, 21.39±0.79, and 24.20±1.18 U/mg (P<0.0001), respectively, the CAT activity was 3.45±0.16, 3.42±0.08, 2.11±0.15, and 3.56±0.02 U/mg (P<0.0001), respectively, and the oxidation product MDA level was 23.03±2.54, 24.95±1.63, 48.10±3.73, and 31.35±4.34 μM/mg (P<0.0001), respectively, suggesting that the restoration of oxidation resistance capacity in BEAS-2B cells can reduce the production of oxidation products. The above results indicate that JAC4 reduces the X-ray-induced DNA damage and apoptosis of BEAS-2B cells by increasing the activities of antioxidases SOD and CAT and reducing the production of MDA and ROS.

The production of ROS may lead to the activation of NF-κB, promote the translocation of NF-κB to the cell nucleus, and mediate various inflammatory responses. The nuclear and cytoplasmic proteins in BEAS-2B cells in all groups were separated. The results of Western blot in FIG. 35 show that the NF-κB expression in the cytoplasm was substantially unchanged in all groups, the NF-κB expression in the nucleus was increased after X-ray treatment, and the increased NF-κB nuclear expression induced by X-ray was inhibited by JAC4 in combination with X-ray treatment. As can be seen from the results in FIG. 35, X-ray may cause NF-κB translocation from the cytoplasm to the nucleus, and JAC4 in combination with X-ray prevents the NF-κB translocation. The above results indicate that JAC4 inhibits the inflammatory responses mediated by the NF-κB nuclear translocation.

To clarify that JAC4 in combination with X-ray exerts synergistic and toxicity-alleviating effects in SPCA-1 and BEAS-2B cells by activating JWA, JWA-knockdown plasmids were transfected into SPCA-1 and BEAS-2B cells.

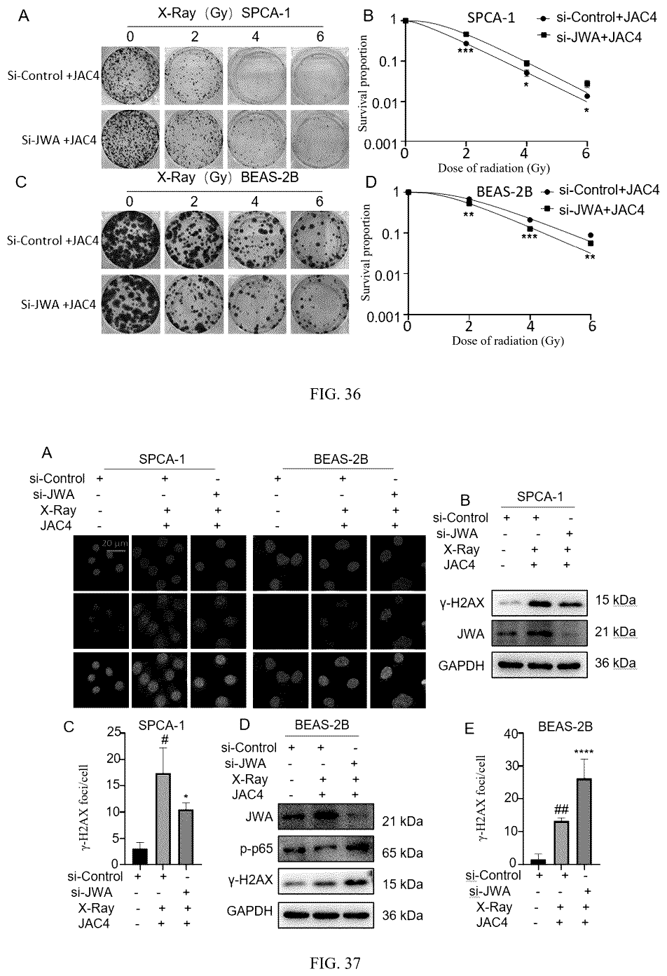

The plate colony formation experiment results, as shown in FIG. 36, show that the number of colonies formed by SPCA-1 cells transfected with JWA-knockdown plasmids was significantly higher after JAC4 treatment, as compared with JAC4-treated non-knockdown si-Control group; A radiosensitization curve was fitted using the one-click multi-target model, which indicates that when comparing JAC4-treated SPCA-1 cells transfected with JWA-knockdown plasmids with the JAC4-treated si-Control group, the cell survival rate of SPCA-1 cells was significantly increased, and when comparing JAC4-treated BEAS-2B cells transfected with JWA-knockdown plasmids with the JAC4-treated non-knockdown si-Control group, the cell survival rate of BEAS-2B cells was significantly decreased. The results further demonstrate that JAC4 exerts the synergistic and toxicity-alleviating effects in X-ray treatment of NSCLC by activating JWA. As can be seen from the results of immunofluorescence study in FIG. 37, when SPCA-1 and BEAS-2B cells were transfected with JWA-knockdown plasmids and treated, 48 h after the transfection, with X-ray for 2 h: the nuclear γ-H2AX fluorescence intensity and γ-H2AX protein expression in SPCA-1 cells transfected with si-Control plasmids were significantly increased after radiation as compared with those in the si-Control group, and the 7-H2AX fluorescence intensity and γ-H2AX protein expression in cells transfected with JWA-knockdown plasmids were significantly reduced after radiation as compared with those in the SPCA-1 cells transfected with si-Control plasmids, indicating that JWA plays a role in tumor cell killing; the nuclear γ-H2AX fluorescence intensity and γ-H2AX protein expression in BEAS-2B cells transfected with si-Control plasmids were significantly increased after radiation as compared with those in the si-Control group, and the γ-H2AX fluorescence intensity and γ-H2AX protein expression in cells transfected with JWA-knockdown plasmids were higher after radiation than those in the SPCA-1 cells transfected with si-Control plasmids, indicating the importance of JWA in protecting normal cells. The above results all show that the deletion of JWA can reduce the synergistic and toxicity-alleviating effects on X-ray in SPCA-1 and BEAS-2B cells.

In summary, it is demonstrated in the present disclosure that JAC4 differentially regulates the X-ray-induced DNA double-strand breaks and apoptosis in SPCA-1 and BEAS-2B cells. In SPCA-1 cells, JAC4 activated JWA to increase the X-ray-induced 7-H2AX and apoptotic cell count; in BEAS-2B cells, γ-H2AX and the apoptotic cell count were significantly reduced; JAC4 must exert the synergistic and toxicity-alleviating effects on X-ray radiation by activating JWA. Compared with the NC group, the colony forming efficiency of SPCA-1 cells transfected with JWA-knockdown plasmids was significantly increased, while the colony forming efficiency of BEAS-2B cells transfected with JWA-knockdown plasmids was significantly reduced. Compared with the NC control group, the DSBs and apoptotic cell count in SPCA-1 cells transfected with JWA-knockdown plasmids were reduced, while the DSBs and apoptotic cell count in BEAS-2B cells transfected with JWA-knockdown plasmids were increased. In addition, JAC4 in combination with X-ray exerts synergistic and toxicity-alleviating effects on subcutaneous NSCLC tumor-bearing model mice. In the subcutaneous tumor-bearing model, the tumor inhibition rate in the JAC4+X-ray group was significantly higher than that of the X-ray monotherapy group; in addition, the combination group exhibited significantly reduced inflammatory cell infiltration in lung tissues, an increased level of anti-inflammatory factor IL-10, and reduced mRNA levels of proinflammatory factors IL-1β, TNF-α, and TGF-β1.

### Example 23. Alleviation of X-Ray-Induced Disruption of Integrity of Single-Cell-Layer Intestinal Epithelium by R-JAC4

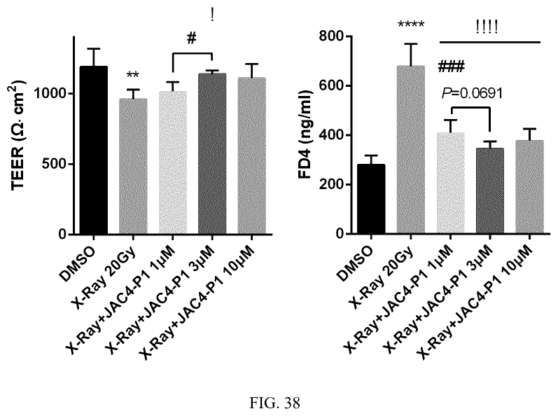

Caco-2 cells were cultured in a mixture of 78% MEM+20% FBS+1% P/S+1% NEAA and seeded in a 24-well chamber at a density of 10,000 cells/well, with the culture medium refreshed once every 2 days until day 11. The cells were then pretreated with DMSO or 1, 3, or 10 μM R-JAC4 for 3 days. The single cell layer model was constructed during the 14 days. The model was then irradiated at a dose of 0 Gy or 20 Gy at a dose rate of 2.5 Gy/min, and the electric resistance of the single cell layer and the permeability of FD4 were measured 24 h later. The results are shown in FIG. 38.

The results show that the electric resistance of the single Caco-2 cell layer membrane was significantly reduced (960.70±67.94 vs 1189±128.9 Ω·cm²) in the radiation group as compared with that in the DMSO group, and the permeability of FD4 was significantly increased (678.90±91.26 vs 280.3±37.40 ng/mL), suggesting that the integrity of the single cell layer was damaged by the 20 Gy radiation; compared with the radiation group, the permeability of FD4 through the single cell layer after radiation was significantly improved by 1, 3, and 10 μM R-JAC4 (410.60±50.99, 345.40±29.85, and 377.30 f 48.24 ng/mL), and the decrease in electric resistance was reduced (1016±64.65, 1138±25.97, and 1109±101.20 Ω·cm²), demonstrating that the R-JAC4 has a protective effect from damage to the integrity the single Caco-2 cell layer caused by radiation.

1. (canceled)

2. (canceled)

3. (canceled)

4. (canceled)

5. (canceled)

6. (canceled)

7. (canceled)

8. (canceled)

9. A method for preventing or treating a radiation-induced injury, comprising: administering to a mammal, preferably a human, in need a therapeutically effective amount of a JWA gene agonist or a pharmaceutical composition thereof.

10. (canceled)

11. A JWA gene agonist or a pharmaceutical composition thereof for preventing or treating a radiation-induced injury.

12. (canceled)

13. A method for preventing or treating a cancer in a mammal, comprising: administering to a mammal, preferably a human, in need a JWA gene agonist or a pharmaceutical composition thereof and a radiation therapy.

14. (canceled)

15. (canceled)

16. The method according to claim 13, wherein the cancer is selected from lung cancer.

17. The method according to claim 16, wherein the cancer is selected from non-small cell lung cancer, preferably lung adenocarcinoma.

18. (canceled)

19. The method according to claim 13, wherein the JWA gene agonist is selected from a compound of formula (I) or a pharmaceutically acceptable salt thereof,

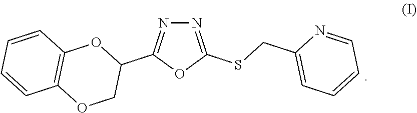 [SMILES: c1ccc(CSc2nnc(C3COc4ccccc4O3)o2)nc1]

20. The method according to claim 19, wherein the compound of formula (I) is selected from one of 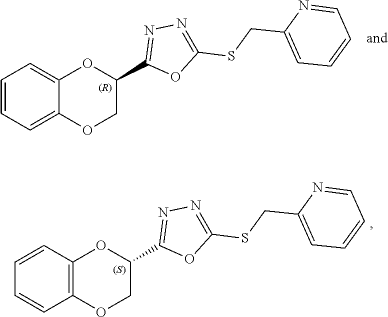
and a combination of the two.

21. (canceled)

22. The method, according to claim 13, wherein the pharmaceutical composition comprises the JWA gene agonist and a pharmaceutically acceptable excipient.

23. The method according to claim 9, wherein the radiation-induced injury is selected from one or two of a radiation-induced intestinal injury and a radiation-induced lung injury.

24. The method according to claim 23, wherein the radiation-induced intestinal injury is radiation enteritis; and/or wherein the radiation-induced lung injury is radiation pneumonitis.

25. The method according to claim 9, wherein the radiation-induced injury is selected from one or two of a radiation oxidative stress injury and a radiation DNA injury.

26. The method according to claim 9, wherein the radiation-induced injury is selected from an X-ray radiation-induced injury.

27. The method according to claim 9, wherein the JWA gene agonist is selected from a compound of formula (I) or a pharmaceutically acceptable salt thereof,

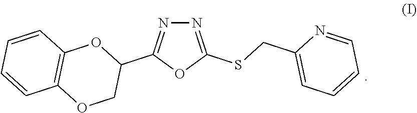 [SMILES: c1ccc(CSc2nnc(C3COc4ccccc4O3)o2)nc1]

28. The method according to claim 27, wherein the compound of formula (I) is selected from one of 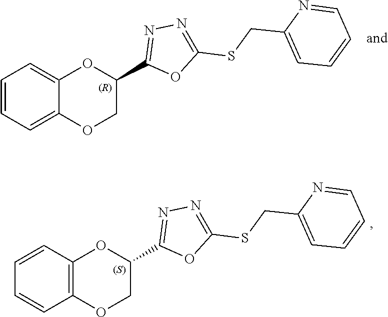
and a combination of the two.

29. The method according to claim 9, wherein the pharmaceutical composition comprises the JWA gene agonist and a pharmaceutically acceptable excipient.

30. The JWA gene agonist or a pharmaceutical composition thereof according to claim 11, wherein the radiation-induced injury is selected from one or two of a radiation-induced intestinal injury and a radiation-induced lung injury.

31. The JWA gene agonist or a pharmaceutical composition thereof according to claim 30, wherein the radiation-induced intestinal injury is radiation enteritis; and/or wherein the radiation-induced lung injury is radiation pneumonitis.

32. The JWA gene agonist or a pharmaceutical composition thereof according to claim 11, wherein the radiation-induced injury is selected from one or two of a radiation oxidative stress injury and a radiation DNA injury.

33. The JWA gene agonist or a pharmaceutical composition thereof according to claim 11, wherein the radiation-induced injury is selected from an X-ray radiation-induced injury.

34. The JWA gene agonist or a pharmaceutical composition thereof according to claim 11, wherein the JWA gene agonist is selected from a compound of formula (I) or a pharmaceutically acceptable salt thereof, 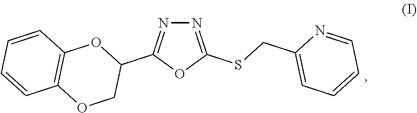 [SMILES: c1ccc(CSc2nnc(C3COc4ccccc4O3)o2)nc1]
preferably, wherein the compound of formula (I) is selected from one of 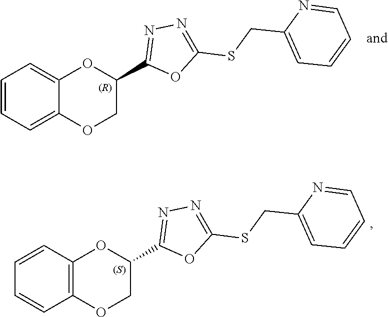
and a combination of the two.
